(19) United States
(12) Patent Application Publication
(10) Pub. No.: US 2016/0022168 A1
(43) Pub. Date: Jan. 28, 2016

(54) BRAIN STATE DEPENDENT THERAPY FOR IMPROVED NEURAL TRAINING AND REHABILITATION

(71) Applicant: UNIVERSITY OF LETHBRIDGE, Lethbridge (CA)

(72) Inventors: Artur Luczak, Lethbridge (CA); Bryan Kolb, Lethbridge (CA); Aaron J. Gruber, Lethbridge (CA); Bruce L. McNaughton, Lethbridge (CA)

(73) Assignee: UNIVERSITY OF LETHBRIDGE, Lethbridge (CA)

(21) Appl. No.: 14/808,411

(22) Filed: Jul. 24, 2015

Related U.S. Application Data
(60) Provisional application No. 62/028,640, filed on Jul. 24, 2014.

(51) Int. Cl. A61B 5/0484; A61N 1/36

(52) U.S. Cl. CPC A61B 5/04842; A61B 5/04845; A61B 5/04847; A61N 1/36139; A61N 1/36067; A61N 1/36082

(57) ABSTRACT

The disclosure provides an apparatus and method for assessing brain plasticity by measuring electrical brain biomarkers, for example, with a near real-time analysis of electrical brain biomarkers, where an increase or decrease in at least one biomarker is indicative of a state of brain plasticity in response to a stimulus or treatment. Brain plasticity can be measured with or without an added stimuli, for example, to determine the best time for learning. Also provided is a method for treating a neurological disease or trauma by applying an electrical or drug stimulus to a patient, where the stimulus is increased or decreased depending on the changes of electrical brain biomarker of the patient. This treatment can occur in near real-time, so a course of treatment can be tailored immediately to a patient's needs.

### RELATED APPLICATIONS

This application claims priority under 35 U.S.C. §119(e) to U.S. Provisional Patent Application No. 62/028,640 filed Jul. 24, 2014, which is incorporated by reference.

### FIELD OF THE INVENTION

The present disclosure relates generally to an apparatus and methods to improve cognitive learning and to treat neurological disorders. More specifically, the present disclosure provides a method for assessing brain plasticity and applying a stimuli during times of high brain plasticity to improve learning, and also for recovery from a neurological injury.

### BACKGROUND OF THE INVENTION

Brain plasticity generally refers to the ability of the brain to modify its own structure and function in response to changes within the body or from the external environment. The area of the brain known as the cortex is especially able to make such modifications.

Brain plasticity triggers normal brain function such as our ability to learn and modify our behavior. This phenomenon is strongest during childhood, but remains a fundamental and significant lifelong property of the brain. Moreover, adult brain plasticity has been clearly implicated as a means for recovery from sensory-motor deprivation, peripheral injury, and brain injury. Brain plasticity has also been implicated in alleviating chronic pain and the development of the ability to use prosthetic devices such as robotic arms for paraplegics, or artificial hearing and visual devices for the deaf and blind, respectively.

Brain plasticity has also been implicated in the relief of various psychiatric and neurodegenerative disorders both in humans and in non-human models. These disorders include, for example, depression, compulsion, psychosocial stress, Alzheimer's disease, and Parkinson's disease. Furthermore, research suggests that the pathology of some of these disorders is associated with the loss of plasticity. Collectively, there is a growing recognition that brain plasticity plays a fundamental role treating psychiatric and degenerative brain disorders.

Recent research has shown the brain is not a passive receiver but must be actively engaged in learning or other activity to maximize plasticity processes. Similar plasticity processes have to be engaged for successful learning and rehabilitation. Unfortunately, it is still not understand how to most effectively enhance plasticity processes to aid “relearning” during rehabilitation. One particularly puzzling aspect of plasticity is that it can be enhanced by activating variety of different molecular mechanisms using diverse pharmacological and non-pharmacological treatments, which treatments seems to have very little in common.

The brain also exhibits complex self-generated and often rhythmic activity patterns. This self-organized activity is most prominent during slow-wave sleep where spontaneous cortical activity is dominated by low-frequency (<1 Hz) oscillatory patterns consisting of an alternation between UP states (burst of population activity), interspersed with DOWN states of generalized silence. This type of brain activity is called synchronized (due to synchronized bursts of activity). On the other extreme is so-called desynchronized brain activity that is characteristic for an awake, attentive human or animal.

Accordingly, it may be favourable to detect or to stimulate a desynchronized brain state where an individual is in a more attentive-like state, thereby promoting learning function and brain plasticity. The detection and stimulation of a desynchronized brain state may also promote brain recovery from neurological disease or other traumatic brain trauma.

Therefore, there is a need to determine when the brain is a desynchronized brain state to promote learning and healing. Therapy designed to promote recovery from various conditions would benefit from detecting when a desynchronized brain state in near real-time, as well as one able to provide for a method of promoting learning ability and brain healing by detecting and increasing the desynchronized brain state in an individual through stimulation.

### SUMMARY OF THE INVENTION

The disclosure provides a novel process for improved efficacy of neural training in healthy subjects and rehabilitation in subjects with neurological disorders. In addition, the invention provides an improved method for the rapid assessment of pharmacological and non-pharmacological rehabilitation and treatment of neurological disorders.

The present disclosure has several commercial applications. Rehabilitation centers can use devices based on this technology to measure brain responsiveness for therapy that will improve rehabilitation following brain damage (e.g. stroke, trauma). In addition, pharmacological companies can benefit from this technology by improving drug screening processes, where drugs most activating to the brain have largest chance to provide benefit. Lastly, a consumer version of the device described herein can be used for the general public for the purpose of using brain activation measures to focus learning/training to times of maximal brain receptivity, and as a biofeedback device for self-training to produce plastic brain states.

Because global brain activity (e.g., level of brain activation) can be easily assessed, for example, using non-invasive electroencephalography (EEG), the apparatus and methods described herein can provide an immediate measure of how “responsive” a patient is for a specific therapy. Previously, there were no standard means of predicting how a given patient will respond to different treatments at different time periods, thus measuring features of ongoing brain activity will allow for a rapid personalization of treatment where timing, type and dosage is chosen based on brain activity to maximize therapeutic benefit. Accordingly, a simple EEG-based device can be used to access brain activity as a marker of brain responsiveness to particular treatment.

In another aspect, developing new drug treatments to improve functional recovery from brain trauma (e.g., stroke rehabilitation) is an exceptionally long and inefficient process requiring months or even years of animal trials because therapeutic value is assessed by behavioral improvement, which is a slow process. The apparatus and methods described herein provide a rapid readout of neural activity underlying treatment related neuronal reorganization and can therefore speed the research and development of new therapies. This means that one can predict the effect of a drug on various conditions, for example, stroke recovery, by measuring only how a therapy activates the brain in acute preparation. This can provide a much more efficient method of screening potential new therapeutic compounds. Accordingly, the invention can be used to greatly reduce the time and costs of screening new therapies.

In one embodiment, the invention provides for an apparatus for assessing brain plasticity by measuring electrical brain biomarkers comprising 1) at least one biosensor to measure at least one electrical brain biomarker; 2) the at least one sensor being operably coupled to at least one measuring device; 3) the at least one measuring device recording the at least one electrical brain biomarkers; 4) the at least one measuring device being operably coupled to at least one processor, the at least one processor giving a near real-time analysis of the at least one electrical brain biomarker, the near real-time analysis being an increase or decrease in the at least one electrical brain biomarker, where a course of treatment can be based on the increase or decrease of the at least one electrical brain biomarker. In some embodiments, the measurement of electrical brain biomarkers is by EEG.

In one embodiment, the measured electrical brain biomarker is indicative of a desynchronized brain state. The desynchronized brain state can be characterized by an increase in power spectra of brain electrical activity in gamma-band and a decrease in power spectra in low frequency brain waves. In some embodiments, the state of brain plasticity can be described by or determined by the ratio of power spectra output in the range of about 40 Hz to about 60 Hz to the power spectra output in the range of about 1.5 Hz to about 3.5 Hz. A higher ratio is indicative of a higher degree of brain plasticity, or a higher level of brain desynchronization. In such cases of higher brain plasticity, the subject can show better response to a stimuli, which can lead to faster learning or faster recovery from a traumatic brain injury or other adverse condition described herein.

In some embodiments, the measuring device can be an electroencephalography (EEG), a magneto-encephalography or a functional magnetic resonance imaging or intracranial recording of neuronal activity. In some embodiments, the processor can be a computer or another device that contains a microprocessor. The computer or microprocessor can also contain software adapted to give a near real-time analysis of the electrical brain biomarker.

In another embodiment, the invention provides methods for assessing brain plasticity by measuring electrical brain biomarkers comprising applying 1) at least one biosensor to a patient, the at least one biosensor measuring at least one electrical brain biomarker; 2) the measurement of the at least one electrical brain biomarker being received by at least on measuring device, the measuring device recording the measurement of the at least one electrical brain biomarker; 3) sending the recording of the measurement of the at least one electrical brain biomarker to at least one processor, the at least one processor giving a near real-time analysis of the at least one electrical brain biomarkers, the near real-time analysis being an increase or decrease in the at least one electrical brain biomarker, where the increase or decrease of the at least one electrical brain biomarker is indicative of a state of brain plasticity in the patient in response to a stimulus. In some embodiments, the measurement of electrical brain biomarkers is by EEG.

In one embodiment, the measured electrical biomarker is indicative of a desynchronized brain state. The desynchronized brain state can be characterized by an increase in power spectra of brain electrical activity in gamma-band and a decrease in power spectra in low frequency brain waves. In some embodiments, the electrical brain biomarker is determined by the ratio of power spectra output in the range of about 40 Hz to about 60 Hz to the power spectra output in the range of about 1.5 Hz to about 3.5 Hz, wherein a larger ratio is indicative of a desynchronized brain state.

In another embodiment, the stimulus is continued if there is an increase in the patient's desynchronized brain state and where the stimulus is discontinued if there is a decrease in the patient's desynchronized brain state.

In another embodiment, the stimulus is an electrical current applied to the patient. In another embodiment, the stimulus is a drug administered to the patient. In yet another embodiment, the stimulus is selected from the group consisting of a visual, auditory, touch, taste or smell where the stimulus is continued if there is an increase in the patient's desynchronized brain state and discontinued if there is a decrease in the patient's desynchronized brain state.

The disclosure further provides for a method for treating a neurological disorder, mental disorder or trauma by measuring electrical brain biomarkers comprising applying at least one biosensor to a patient, the at least one biosensor measuring at least one electrical brain biomarker; and the measurement of the at least one electrical brain biomarker being received by at least on measuring device, the measuring device recording the measurement of the at least one electrical brain biomarker; and sending the recording of the measurement of the at least one electrical brain biomarker to at least one processor, the at least one processor giving a near real-time analysis of the at least one electrical brain biomarker, the near real-time analysis being an increase or decrease in an electrical brain biomarker, where a course of treatment is based on the increase or decrease of an electrical brain biomarker.

In one embodiment, the measured electrical biomarker is indicative of a desynchronized brain state. The desynchronized brain state can be characterized by an increase in power spectra of brain electrical activity in gamma-band and a decrease in power spectra in low frequency brain waves. In some embodiments, the electrical brain biomarker is determined by the ratio of power spectra output in the range of about 40 Hz to about 60 Hz to the power spectra output in the range of about 1.5 Hz to about 3.5 Hz, wherein a larger ratio is indicative of a desynchronized brain state.

In another embodiment, the course of treatment is continued if there is an increase in the patient's desynchronized brain state and where the course of treatment is discontinued it there is a decrease in the patient's desynchronized brain state. The course of treatment can be an electrical stimulus or a drug regimen. The type, dosage and duration of drug administered to the patient can be changed based on an increase or decrease in the desynchronized brain state.

In another embodiment, the disease treated is a neurological or mental disorder. In another embodiment the disease treaded is a neurodegenerative disease. In a further embodiment, the brain trauma treated is a stroke. In various embodiments discussed above and herein below, the patient can be a human, or a non-human, animal.

### BRIEF DESCRIPTION OF THE DRAWINGS

The patent or application file contains at least one drawing executed in color. Copies of this patent or patent application publication with color drawings will be provided by the Office upon request and payment of the necessary fee.

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-F illustrates experimental protocol for somatosensory stimulation, and synchronized and desynchronized brain states. (A) Example LFP in S1 under urethane anesthesia. The grey shaded area indicates the period of tactile stimulation consisting of 1 sec long periods of vibration at 20 Hz (inset). (B) Example LFP in S1 under urethane anaesthesia after injection of amphetamine. (C) Example LFP and unit activity under urethane anaesthesia shown at a higher temporal resolution. Note prominent UP and DOWN states characteristic of the synchronized brain state. (D) Example LFP and unit activity after amphetamine injection. Note that in the desynchronised state, fluctuations of LFP and unit activity are of smaller amplitude. (E) Mean stimulus-triggered LFP across animals in S1 in urethane only and urethane+amphetamine conditions. (F) The same as (E) for average spiking activity.

FIG. 2A-G illustrates similarity between spontaneous and evoked temporal patterns is enhanced by amphetamine. (A) The latency of a neuron is defined as the center of mass of the cross-correlogram of the neuron with the summed activity of all other simultaneously recorded cells (MUA). (B) Example of temporal patterns before, during, and after tactile stimulation in amphetamine condition from a representative experiment. Each row represents a cross-correlogram of a neuron with the summed activity of all other neurons, normalized between 0 and 1. Neurons are ordered according to their latency (red dot) during stimulation (middle panel). The same order was used to plot the latency of the neurons before stimulation (top panel) and after stimulation (bottom). (C) Left: Scatter plot of the latencies of neurons for evoked activity and spontaneous activity before stimulation plotted for the same neurons as in (B). Right: Scatter plot of the latencies for the same neurons for evoked and spontaneous activity after stimulation. Note that the distribution of points is closer to the identity line, indicating higher similarity between latencies for evoked and spontaneous period after stimulation. (D) Similarity of spontaneous activity patterns to evoked activity patterns (quantified by latency correlation) before (blue/left) and after (orange/right) tactile stimulation, for amphetamine (left graph) and urethane-only (right graph) conditions. Connected dots represent one animal. Note that in the amphetamine condition, the similarity of spontaneous sequences to evoked sequences increases for all rats after stimulation. Rats that show latency correlation increase under urethane are shown in magenta, and rats that show latency correlation decrease under urethane are depicted with light blue lines. (E) Average change in similarity to evoked patterns, based on amphetamine and urethane only data from panel (D); red (left for ure+amph) and blue (left for ure only) bars, respectively. White (unfilled) bars show average change in similarity to evoked patterns calculated from pair-wise cross-correlograms. (F) Synchronized brain state reduces formation of reverbatory activity in somatosensory cortex. X-axis shows the percentage of time that the population activity spent in DOWN states. Rats that show latency correlation increase under urethane (Ureth▴) are marked with magenta color, and rats that show latency correlation decrease under urethane (Ureth▾) are shown in light blue. Red color denotes rats after amphetamine injection. (G) Latency correlation evolution in time in 51: before, during and after stimulation (each dot represents the average from all rats; error bars denote SEM). The shaded area corresponds to the stimulation period. The insets at the top show the slope distribution of latency correlations in the corresponding period of the experiment for each rat. Note that the spontaneous activity becomes gradually more similar to evoked patterns during stimulation, and how the similarity slowly decreases after stimulation. Stars denote points significantly different from spontaneous activity before stimulation (p<0.05, t-test).

FIG. 3A-E illustrates template matching. (A) Raster plot of representative 2 sec of spontaneous activity before tactile stimulation. Blue traces at the top of rasters show matching score, and stars indicate ‘good matches’ defined as above 95% of matching score. (B) Raster plot of 2 sec of spontaneous activity after tactile stimulation. Note the greater number of good matches (denoted by stars) after stimulation than before. (C) Stimulus-triggered activity used as a template. In Figure A and B, template is superimposed on sample activity windows showing good match. (D) Example of template matching histograms for representative data from one rat injected with amphetamine. The solid blue line (narrower curve) shows the frequency of the matching values before stimulation. The solid orange line (broader curve) shows the frequency of the matching values after stimulation. The shaded regions denote the 0.1% of the highest matching values. The dotted lines represent the mean of such values. (E) Box plot of differences between the highest matching values for spontaneous activity after and before stimulation, which corresponds to the differences between the dotted lines in (D), for all rats. On each box, the central mark denotes the median, the edges of the box are the 25th and 75th percentiles, and the whiskers extend to the most extreme data points. In the amphetamine condition, the template matching between spontaneous and evoked patterns increases after stimulation, which is consistent with latency analyses shown in FIG. 2.

FIG. 4A-C illustrates persistence of firing rate correlation in S1. (A) Pair-wise firing rate correlation matrices for the neurons in S1 before, during and after tactile stimulation for a representative animal injected with amphetamine. To facilitate visual comparison of the matrices, the elements were sorted to group together neurons with similar correlations (for sorting, Applicant used values of the first principal component calculated for the matrix from the stimulation period). The same order was used for the other matrices. (B) Scatter plot of the similarities (measured as the Euclidean distance) between firing rate correlation matrices for each animal. Distribution of points above the identity line indicates that spontaneous firing rate correlations after stimulation become more similar to stimulus-evoked correlations. Inset shows the distribution of differences between distances in the corresponding scatter plots (D denotes Euclidian distance). (C) Changes in firing rate correlations over time, analyzed with explained variance (EV) in the amphetamine condition. The grey shaded area corresponds to the stimulation period. The error bars correspond to the SEM. Solid lines represent the explained variance and dotted lines represent the reversed explained variance (rev-EV, see Supplemental Experimental Procedures). Stars indicate points significantly different from rev-EV (control) values (p<0.05, t-test)

FIG. 5A-H illustrates experimental protocol for auditory experiments. (A-D): Example LFP (top) and unit activity (bottom) in auditory cortex under urethane anaesthesia alone (A), after infusion of carbachol (B), amphetamine injection (C), and NMDA antagonist injection (D). Grey shaded area indicates the period of auditory stimulation consisting of 500 ms long tones interspersed with 1 sec of silence (inset). (E) Same type of plot as (A-D) for awake, head restrained rat. (F) Brain state in each condition measured as % of down states duration. (G) Stimulus-triggered LFP in A1, averaged across all animals for each experimental condition. The bars on top represent the stimulation duration for the awake and anaesthetized cases respectively. (H) The same as (G) for average spiking activity.

FIG. 6A-B. (A) An example of neuronal spiking patterns during different levels of brain synchronization. Data recorded in auditory cortex in awake, passively listening rat. (Top) Brain activity shows synchronized bursts (UP states) interspersed with periods of neuronal silence. During desynchronized brain activity, spiking patterns show weaker global fluctuations and more continues firing. The raster shows spikes of simultaneously recorded neurons and the blue trace shows local field potential. At bottom is the multiunit firing rate (MUA). Note that there is continuum of brain synchrony levels. (B) LFP power spectra in synchronized and desynchronized states. Note that low frequency (<˜20 Hz) power is higher in synchronized states, whereas gamma frequency (50-80 Hz) power is slightly higher in desynchronized states.

FIG. 7A-D illustrates cortical plasticity is enhanced in desynchronized brain state. (A) Example of temporal patterns before, during, and after tactile stimulation in amphetamine condition from a representative experiment. Each row represents a cross-correlogram of a neuron with the summed activity of all other neurons, normalized between 0 and 1. Neurons are ordered according to their latency (red dot) during stimulation (middle panel). The same order was used to plot the latency of the neurons before stimulation (top panel) and after stimulation (bottom). Note that after stimulation, spontaneous pattern become more similar to stimulus evoked patterns. (B) Similarity of spontaneous patterns to evoked patterns throughout experiment duration (average from all rats; error bars denote SEM). The shaded area corresponds to the stimulation period. Note how the spontaneous activity becomes gradually more similar to evoked patterns during stimulation, and how the similarity slowly decreases after stimulation. Stars denote points significantly different from spontaneous activity before stimulation (p<0.05, t-test). (C) Average change in similarity to evoked patterns in urethane, carbachol, amphetamine, and awake conditions. More positive values indicate bigger changes (plasticity) in spontaneous patters following stimulation. (D) Brain synchrony in each condition measured as % of UP states duration. Note that more desynchronized brain activity correlates with higher plasticity. (Figures modified form Bermudez et al. 2013).

FIG. 8 illustrates the outline of a method to treat a patient to increase learning function or promote healing from a brain injury.

FIG. 9A-E illustrates the effect of tDCS on brain activity. (A) On the left: example of tDCS electrode placement for stimulation of frontal cortex in human subject (red color denotes anode and blue denotes cathode). On the right: the polarity of stimulation causes differential effects on neuronal activation. Anodal stimulation increased spiking activity whereas cathodal stimulation has opposite effect. (B) Representative coronal sections (40 μm) of the brain of an animal with ischemic stroke. Localization of the stroke area (red) corresponds to the primary motor cortex. Top section shows silicon probe traces (pink) in the deeper layers of the limb somatosensory cortex in both hemispheres. (C) Illustration of tDCS electrodes position, location of photothrombotic lesion (stroke label), and areas with inserted silicon probes (green boxes; schematic of the silicon probe with 8 shanks is on right side). (D) Difference in LFP power spectra: after minus before tDCS stimulation in anesthetized rats. Blue trace (broad line) shows average and standard error (shaded area) across all animals. Green (middle), red (top) and black (bottom) lines show average for Control (5 rats), Stroke group (6 rats), and Stroke+Stim (in this group tDCS was applied for 2 weeks before recordings; 5 rats) respectively. (E) Change in LFP coherence between hemispheres (red-Stroke group (lowest starting point line); black—Stroke+Stim (highest starting point line); green—intact control group (intermediate starting point line)). Note that for all groups tDCS increased power and coherence in 10-40 Hz band indicating that tDCS increases brain desynchronization.

FIG. 10A-E illustrates changes in LFP after stroke and electrical stimulation. (A) Power spectra in sensory cortex in the Control group (green), the Stroke group (red) and Stroke+Stim group (black). Dashed lines show standard error. For each animal, data was averaged over both hemispheres. (B) Coherence between hemispheres for the same experimental groups as in panel A. Error bars denote standard error. Note the higher coherence in 20-40 Hz band for Control and Stroke+Stim groups. (C) Illustration of definition of offset between homologous areas between hemispheres. Circles represent recording sites. (D) Coherence as a function of distance between homologous areas. Each cross represents an average coherence between 20-40 Hz across animals for each pair of shanks. Colors represent experimental groups as shown in FIG. 10A. Lines show linear fit for each group. For Control and Stroke+Stim groups the coherence decreased with offset between areas. (E) Coherence as a function of distance between shanks within hemisphere. Each cross represents an average coherence between 20-40 Hz across animals for each pair of shanks. Colors represent experimental groups as shown in FIG. 10A. Lines show linear fit for each group. For all groups the coherence decreased with distance between recording shanks.

FIG. 11A-B illustrates animals with a more desynchronized brain state show better recovery from stroke. (A) Picture of a rat performing a skilled reaching movement. (B) Relationship between coherence in gamma range (another marker of desynchronized brain state) and reaching success (green—Control group; red—Stroke group; black—Stroke+Stim group). Dashed line represents the least-squares fit for points combined from all groups.

FIG. 12A-D illustrates changes in brain activity after electrical stimulation. (A) Difference in LFP power spectra: after minus before electrical stimulation. Blue trace shows average and standard error (shaded area) for all animals. Note increase in power after stimulation in 10-40 Hz band. Green, black and red lines show average for Control, Stroke+Stim and Stroke group respectively. (B) Change in LFP power spectra after electrical stimulation in intact (dark blue) and stroke hemisphere (light blue). (C) Increase in LFP coherence after electrical stimulation in 10-40 Hz band. Thick blue line shows average change in coherence across all recording sites for all animals; shaded area shows standard error. Dark blue, light blue and yellow lines represent an average change in coherence within intact hemisphere, stroke hemisphere and between hemispheres, respectively. (D) Change in LFP coherence in stroke hemisphere (red-Stroke group; black—Stroke+Stim) and in control group in left hemisphere (green) (difference between groups were not significant in 10-40 Hz band; p>0.8 for all between groups comparisons; t-test). Note that when brain is in a more desynchronized state (indicated by increase in LFP power in low-gamma frequencies: FIG. 12A, B), there is also an increase in coherence in the low-gamma band (FIG. 12C, D), which indicates that coherence can also be used as measure of brain state and brain plasticity.

FIG. 13 illustrates memory test experiments. Subjects were shown 80 pairs of words and non-words (example is shown in the left panel (“learning phase”)). All word pairs to be memorized were presented consecutively until the subjects viewed all word pairs. More than 30 seconds after the end of the training/learning phase, subjects were tested on their ability to remember presented pairs by selecting one of four possible answers (example in the right panel (“testing phase”)).

FIG. 14A-B. (A) Illustration of a cap with 128 electrodes used for EEG recordings. (B) Summary of results of the study. Based on EEG measurements, Applicant computed brain plasticity index during presentation of every word pair. Next, Applicant computed an average plasticity index for pairs which subject correctly remembered and those that he/she did not (errors). Each line in plot represents data from one participant.

### DETAILED DESCRIPTION

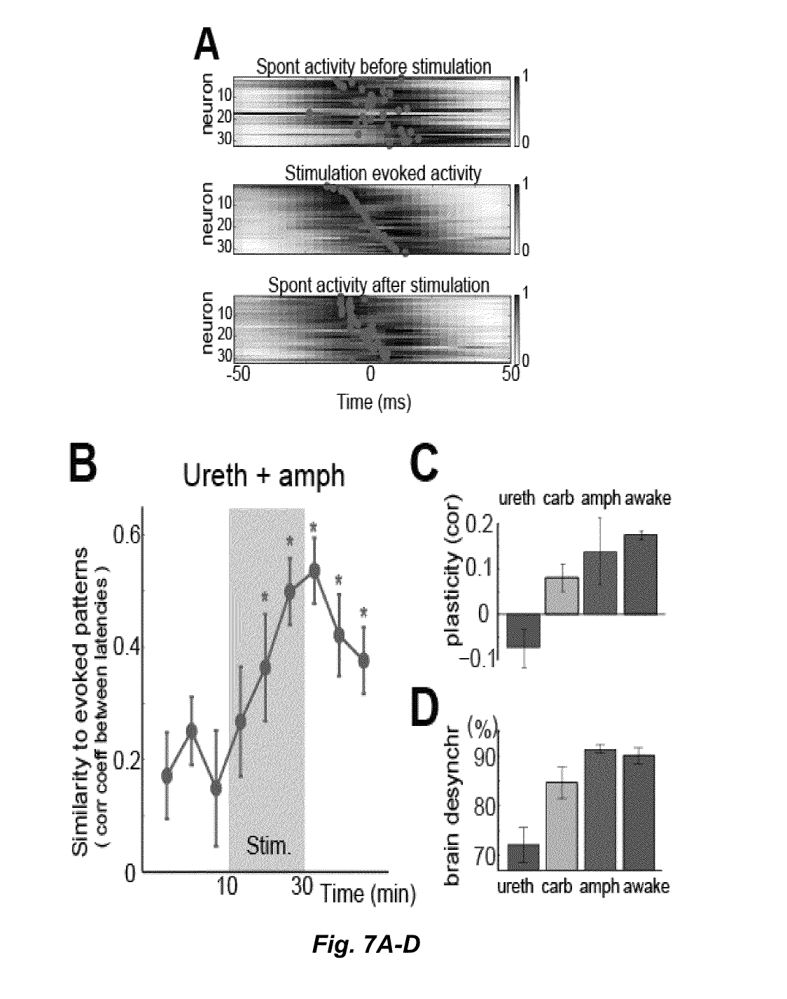

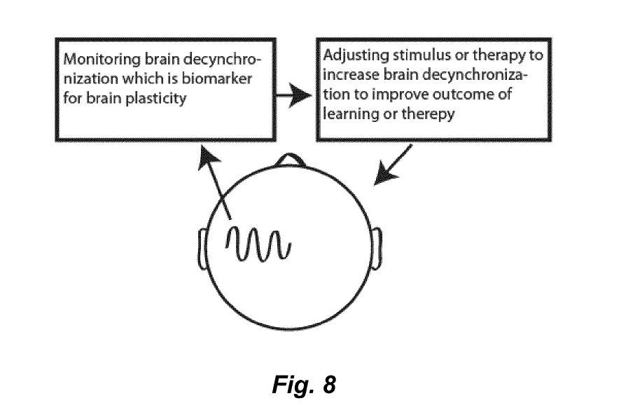

Any learning or rehabilitation requires plastic changes in the brain. Applicant's research shows that the level of plasticity in the brain is related to certain characteristic features of ongoing brain activity (see FIG. 7 C, D). This provides a near real-time means to infer neural plasticity based on, for example, non-invasive measurements (electroencephalography) in humans or in animal subjects. Thus, parameters of any treatment (e.g., dosage, duration of treatment, treatment method) can be quickly adjusted based on instant measurement of brain activity as a proxy measure of brain plasticity. This new process enables refined treatments that maximize plasticity processes and thus maximize treatment effectiveness (see FIG. 8).

Currently, the effectiveness of rehabilitation from brain injury can be assessed only after weeks or months of therapy based on behavioral improvements, thus wasting valuable time if therapy is not effective. Here, Applicant proposes a novel method for measuring plasticity in near-real time by using brain activity as a biomarker, which allows for significant increases in the effectiveness of the many therapies dependent on brain plasticity (e.g. therapy for brain stroke patients). Measuring brain activity can provide instantaneous measure how given therapy drives plasticity processes and thus provide prediction about effectiveness of this therapy. This also allows quick adjustments of therapy based on brain signals to evoke maximal therapeutic effect. No other methods presently allow doctors to judge immediately how effective particular type of therapy will be for a given patient. This technology thus provides processes for personalizing the treatment of neurological disorders for each individual.

In another aspect, developing new drug treatments to improve functional recovery from brain trauma (e.g. stroke rehabilitation) is an exceptionally long and inefficient process requiring months or even years of animal trials because therapeutic value is assessed by behavioral improvement, which is a slow process. In some embodiments, the invention provides a rapid readout of neural activity underlying treatment related to neuronal reorganization and can therefore speed the research and development of new therapies, specifically new drug therapies. This means that it could be possible to predict the effect of a drug on conditions such as stroke recovery by measuring only how it activates the brain in acute preparation. This could provide much more efficient method of screening for potential new therapeutic compounds. Accordingly, the invention can be used to greatly reduce the time and costs of screening new therapies.

In general, brain waves are usually divided into (1) delta waves, having a frequency range of 1.5-3.5 Hz, (2) theta waves, having a frequency range of 3.5-7.5 Hz, (3) alpha waves, having a frequency range of 7.5-12.5 Hz, and (4) beta waves, having a frequency range of 12.5-20 Hz. Some frequencies above 20 Hz, such as the gamma range (around 40-60 Hz), have been implicated in various types of cognitive processing, although their role in indicating overall mood is still unclear.

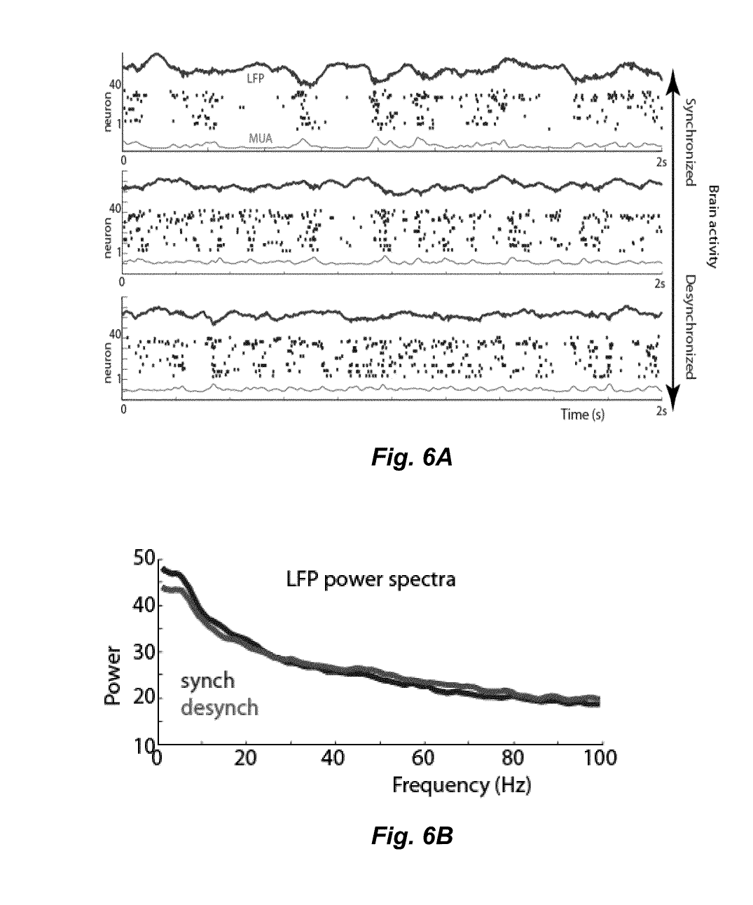

An increase or decrease in the power spectra of the delta, theta, alpha, beta and gamma waves can be used as a biomarker for brain plasticity. Preferably, an electrical brain biomarker is an increase in a patient's desynchronized brain state. As used herein, a “desynchronized brain state” is characterized by an increase in the power output of high frequency (gamma) brain waves and a decrease in the power output in low frequency (delta/theta) brain waves (see FIG. 6B).

In one aspect of the disclosure, an apparatus and method is provided for assessing brain plasticity by measuring electrical brain biomarkers comprising 1) at least one biosensor to measure at least one electrical brain biomarker; 2) the at least one sensor being operably coupled to at least one measuring device; 3) the at least one measuring device recording the at least one electrical brain biomarkers; 4) the at least one measuring device being operably coupled to at least one processor, the at least one processor giving a real-time analysis of the at least one electrical brain biomarker, the real-time analysis being an increase or decrease in the at least one electrical brain biomarker, where a course of treatment is based on the increase or decrease of the at least one electrical brain biomarker. The apparatus can configured to determine a brain plasticity index by calculating the ration of power spectra output of gamma waves (40-60 Hz) as compared low frequency brain waves (1.5-3.5 Hz). A higher brain plastic index is indicative of a more desynchronized brain state.

A sensor may comprise a sensor, sensor electrical components for providing power to the sensor and generating the sensor signal, a sensor communication system for carrying the sensor signal to controller, and a sensor housing for enclosing the electrical components and the communication system. Sensors may be devices configured to sense brain electrical activity and biomarkers. For example, sensors may embody electrodes configured to sense electrical currents created by synaptic potentials. Such sensors are well known in the in the art and may include, for example, epicortical electrodes, deep electrodes, brain electrodes or peripheral electrodes.

In some embodiments, the electrodes each respectively has at least two wires at whose ends a potential difference is applied for the purpose of stimulation. The electrodes can also be microelectrodes or macroelectrodes. Alternatively, the electrodes each can comprise a single wire. In this case for the purpose of the stimulation, respective potential differences are applied between the individual wire and the metallic part of the housing of the generator. Additionally but not obligatory, a potential difference can be measured by means of the electrodes in order to establish a pathological activity. In a further embodiment, the electrodes can each also be comprised of more than two individual wires that can serve both for the determination of measurement signals in the brain as well as for the stimulation. For example, four wires can be provided in a conductor cable, whereby between different ends a potential difference can be applied or can be measured. In this manner the magnitude of the area of the brain from which the signal is derived as well as the area of the brain that is stimulated or the target area can be varied. The number of wires from which the electrodes are made is limited as to its upper value only by the maximum thickness of the cable that is to be inserted into the brain so that the smallest amount of brain matter will be damaged. Commercial electrodes comprise four wires, although five, six, or more wires or only two wires may be comprised in the electrode.

For the case in which the electrodes each comprise more than two wires, at least two of these wires can also function as the sensors so that in this special case an embodiment is provided in which the electrodes and the sensors can be united in a single component. If the electrode is comprised of n wires, a stimulation can be effected over at least one pair of wires so that by the pair formation sub-combinations of wires are possible. Apart from this component additional sensors can be provided which are not structurally united with the electrodes.

In other embodiments, the number of electrodes used are sufficient to measure the desired biomarker. An emphasis on the frontal lobe can aid the accuracy of measurements. Moreover, the methods described herein are compatible with any commercially available sensor apparatus. Such apparatus may contain about 1-5, 1-10, 1-20, 1-50, 1-75, 1-100, 1-125, 1-150, 1-175, or 1-200, or 200 or more electrodes for measuring a biomarker.

Most techniques for measuring the brain activity of a subject have concentrated on the electroencephalogram (EEG) signal. The EEG is an electrical signal that is read on the surface of the skull that reflects the average activity of large groups of neurons and may, if properly interpreted, be indicative of the psychological state of the subject. In general, the lower the mean frequency of the EEG signal, the lower the state of alertness, although many other factors may influence the interpretation of the EEG signal, including the location on the scalp of the EEG readings, the degree of synchronization between readings, and whether any psychological pathology is present.

Although EEG is currently the most cost-effective means of monitoring the dynamic state of the brain for a given space and time resolution, other means are available to person of ordinary skill in the art. The electrical brain biomarker measuring device is monitored by means including, but not limited to EEG, functional magnetic resonance imaging (fMRI), positron emission tomography, magnetoencephalography (MEG), nuclear magnetic resonance spectroscopy, electrocorticography, single-photon emission computed tomography, near-infrared spectroscopy (NIRS), intracranial electrical recordings, and optical imaging, or by any other acceptable monitoring means.

According to the invention, the device is provided with means that can recognize the signals from the measuring device. The means for recognizing and analyzing the electrical brain biomarkers can comprise a computer that processes the electrical signals from the sensors. The computer can then compare the process signal with data stored in the computer.

In some embodiments, a computational system may include quantitative electroencephalogram software for monitoring the electrical biomarker signal and comparing the signal to a desired electrical biomarker signal to determine the difference between the measured signal and the desired signal. In other embodiments, the means for recognizing the signals from the sensors comprises a chip or another electronic device with comparable computing power. As mentioned above, the computational system can calculate in near-real time the ration of power spectra output in the gamma frequency as compared to the power spectra output in the delta frequency. This ratio can be compared to reference or control sample and the stimuli can be increased or decreased based on an increase or decrease in the brain plasticity index. In some embodiments, the control sample is a measurement of electrical brain biomarkers from the test subject prior to the stimuli. In other embodiments, the systems determines an increase or decrease in the desynchronized brains state of the subject.

The recognized electrical brain biomarkers are preferably displayed to the user using well known means such as a monitor or any device capable of displaying the measured data.

In some embodiments, the patient is subjected to an auditory, visual, taste, touch, or smell stimulus to increase brain plasticity. A patient's electrical brain biomarkers can be measured in response to the sensory stimulus. In some embodiments, the electrical brain biomarker is an increase in the desynchronized brain state. If there is an increase in brain desynchronized brain state, then the stimulus is continued and if there is a decrease in the desynchronized brains state, the stimulus is stopped. Examples of sensory stimulation include but are not limited to various intensities of light, color of light, visual pulses, picture images, abstract images, white light, no light, noise, music, speech, tones, pulses, voices, alarms, high pitched frequencies, low pitched frequencies, sweet, salty, acidic, basic tasting substances, hot and cold temperature, soft or hard feeling substances, malodorous or pleasant smelling substances. The stimulus can also be a combination of any of the above sensory stimulus. Moreover, the stimulus may also be an administered drug or electrical stimulation as described below.

In other embodiments, a patient's electrical brain biomarkers are measured to determine if the patient is in a desynchronized brain state. Once it is determined that a desynchronized brain state exists (the brain has more plasticity), the patient may be given tasks or stimulus. Because the brain is in a desynchronized brain state, this may increase the rate at which learning occurs. For example, a student may be more efficient in learning test material when they are in a desynchronized brain state.

As an example, Applicant has shown that using memory task and EEG recordings to simultaneously monitor brain activity, human test subjects were able to memorize (encode) more information when the brain is in a more desynchronized state. Thus, consistent with Applicant's animal studies described herein below, brain plasticity in humans is correlated with brain desynchronization.

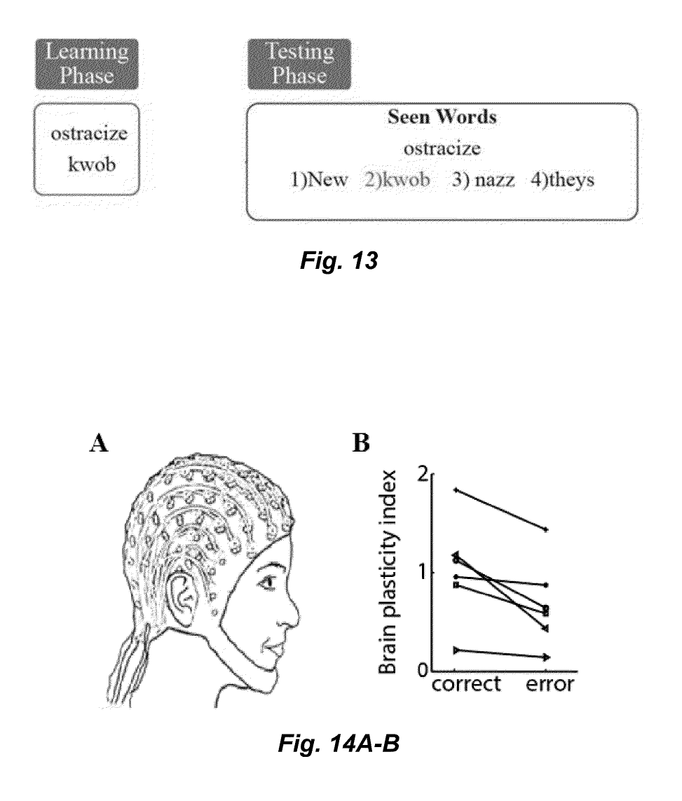

The participants of the memory task performed a word-pair matching task that consisted of a learning phase, which was then followed by a testing phase. There was at least a 30 second interval between learning and testing phases. The learning phase consisted of four blocks of 20 word pairs displayed on a monitor for five seconds, with a one second interval between each word pair. The testing phase was self-paced, where the participants were tasked with matching each word with its corresponding non-word. A desynchronization index was calculated for each presentation of words, which in some embodiments was calculated as the ratio of gamma to delta power spectra output (see FIG. 13). As mention previously, delta waves have a frequency range of 1.5-3.5 Hz, and the gamma range was defined as frequencies of about 40-60 Hz. EEG was recorded with 128 Ag/Ag—Cl electrodes in an elastic net, but any commercially available or custom measuring device can be used. Care was given to placing at least one electrode over the frontal lobe to enhance accuracy of the data. Measured results were statistically significant (p=0.0313; Wilcoxon signed-rank test). Thus, the performance of the participants in word-matching task increases if the word-pairs were presented during a more desynchronized (plastic) brain state.

In other examples, the desychronization index can be calculated as the power output in 2-5 Hz band divided by the power output in 35-53 Hz band and normalized (divided) by total power output in the 1-200 Hz band. Other variations of a higher power over a lower power output can be used in various embodiments, such as a ratio of 1-4 Hz power spectra output to 30-60 Hz power spectra output, or 2-3 Hz power spectra output to 40-50 (or 50-60) Hz power spectra output.

Applicant found that the brain plasticity index had significantly higher values during the presentation of pairs that were later correctly remembered as compared to pairs that were not. Thus, the brain plasticity index calculated based on brain desynchronization is a good predictor of brain plasticity as evaluated by memory task. Therefore, when stimuli is presented when the brain has a higher plasticity index, which is indicative of a desynchronized brain state, the subject shows a better response than when the stimuli is presented when the subject has a lower brain plasticity index, indicative of a lower or non-desynchronized brain state.

Generally, memory can be delineated into short term and long term memory. Short term memory is of a duration of about 30 seconds or less. On the other hand, once information has entered into long term memory, it can persist indefinitely. Accordingly, by varying the time interval between the learning phase and the testing phase, either short term memory or long term memory can be targeted using a method as described herein. Generally, the time between training and testing phase was always more than 30 seconds, thus the brain plasticity index is correlated with long-term memory.

The disclosure also provides for a method for treating neurological and mental disorders, as well as neurological trauma by applying at least one biosensor to a patient, were the patient is a human or non-human animal, the at least one biosensor measuring at least one electrical brain biomarker. The measurement of the at least one electrical brain biomarker is received by at least on measuring device, which records the measurement of the at least one electrical brain biomarker. At least one processor receives the recording and gives a near real-time analysis of the at least one electrical brain biomarker, the near real-time analysis being an increase or decrease in an electrical brain biomarker, where a course of treatment is based on the increase or decrease of an electrical brain biomarker. In some embodiments, the treatment is scored according to a brain plasticity index. This index can be calculated as the ratio between the gamma spectra power output and the delta spectra power output. When a subject has a higher brain plasticity index, indicative of a desynchronized brain state, task performance, such as learning, is increased.

In some embodiments, the neurological disorder or mental disorder includes, for example aboulia, absence epilepsy, acute stress disorder, adjustment disorder, adolescent antisocial behavior, adult antisocial behavior, adverse effects of medication—not otherwise specified, age-related cognitive decline, agoraphobia, alcohol abuse, alcohol dependence, alcohol withdrawal, alcoholic hallucinosis, Alzheimer's disease, amnestic disorder, amphetamine dependence, amphetamine withdrawal psychosis, anorexia nervosa, anterograde amnesia, antisocial personality disorder, anxiety disorder, anxiolytic-related disorders, attention deficit disorder, attention deficit hyperactivity disorder, autophagia, avoidant personality disorder, barbiturate dependence, benzodiazepine dependence, benzodiazepine misus, benzodiazepine withdrawal, bereavement, bibliomania, binge eating disorder, bipolar disorder, bipolar I disorder, bipolar II disorder, body dysmorphic disorder, borderline intellectual functioning, borderline personality disorder, brief psychotic disorder, bulimia nervosa, caffeine-related disorder, caffeine-induced sleep disorder, cannabis dependence, claustrophobia, catatonic disorder, catatonic schizophrenia, childhood amnesia, childhood antisocial behavior, circadian rhythm sleep disorder, cocaine dependence, cocaine intoxication, cognitive disorder, communication disorder, conduct disorder, cotard delusion, cyclothymia, delirium tremens, depersonalization disorder, depressive disorder, derealization disorder, desynchronosis, developmental coordination disorder, diogenes syndrome, dispareunia, dissociative identity disorder (multiple personality disorder), dyslexia, dysthymia, EDNOS, encopresis, Ekbom's Syndrome (Delusional Parasitosis), enuresis (not due to a general medical condition), erotomania, exhibitionism, factitious disorder, Fregoli delusion, Frotteurism, Fugue State, Ganser syndrome (e.g., due to a mental disorder), generalized anxiety disorder, general adaptation syndrome, grandiose delusions, hallucinogen-related disorder, hallucinogen persisting perception disorder, histrionic personality disorder, Huntington's disease, hypomanic episode, hypochondriasis, impulse control disorder, impulse-control disorder not elsewhere classified, inhalant abuse, insomnia due to a general medical condition, intellectual disability, intermittent explosive disorder, kleptomania, Korsakoffs syndrome, lacunar amnesia, major depressive disorder, major depressive episode, male erectile disorder, malingering, manic episode, mathematics disorder, medication-related disorder, melancholia, minor depressive disorder, minor depressive episode, misophonia, mixed episode, mood disorder, mood episode, morbid jealousy, Munchausen's syndrome, Munchausen's syndrome by proxy, narcissistic personality disorder, neglect of child, neuroleptic-related disorder, nicotine withdrawal, night eating syndrome, nightmare disorder, obsessive-compulsive disorder (OCD), obsessive-compulsive personality disorder (OCPD), oneirophrenia, opioid dependence, opioid-related disorder, oppositional defiant disorder (ODD), orthorexia (ON), pain disorder, panic disorder, paranoid personality disorder, parasomnia, Parkinson's Disease, partner relational problem, pathological gambling, perfectionism, persecutory delusion, personality change due to a general medical condition, personality disorder, Phencyclidine (or phencyclidine-like)-related disorder, phobic disorder, phonological disorder, physical abuse, pica, polysubstance-related disorder, post-traumatic embitterment disorder (PTED), posttraumatic stress disorder (PTSD), premature ejaculation, primary hypersomnia, primary insomnia, psychogenic amnesia, psychological factor affecting medical condition, psychotic disorder, pyromania, reactive attachment disorder of infancy or early childhood, reading disorder, recurrent brief depression, relational disorder, residual schizophrenia, retrograde amnesia, rumination syndrome, sadomasochism, schizoaffective disorder, schizoid personality disorder, schizophrenia, schizophreniform disorder, schizotypal personality disorder, seasonal affective disorder, sedative-, hypnotic-, or anxiolytic-related disorder, selective mutism, separation anxiety disorder, severe mental retardation, shared psychotic disorder, sleep disorder, sleep paralysis, sleep terror disorder, sleepwalking disorder, social anxiety disorder, social phobia, somatization disorder, somatoform disorder, specific phobia, Sendhal syndrome, stereotypic movement disorder, stuttering, substance-related disorder, Tardive dyskinesia, Tourette syndrome, transient global amnesia and trichotillomania.

In another embodiment, the neurological or mental disorder is a neurodegenerative diseases or conditions result in progressive degeneration and/or death of nerve cells which causes problems with movement (called ataxias), or mental functioning (called dementias) including, but not limited to Alzheimer's disease, Pick's disease, corticobasal degeneration, progressive supranuclear palsy, frontotemporal dementia, parkinsonism (linked to chromosome 17, FTDP-17), Parkinson's disease, diffuse Lewy body disease, brain stroke, amyotrophic lateral sclerosis, Niemann-Pick disease, Hallervorden-Spatz syndrome, Down syndrome, neuroaxonal dystrophy, and multiple system atrophy.

In other embodiments, the neurological trauma is a traumatic brain injury. Traumatic brain injury is defined as damage to the brain resulting from external mechanical force, such as rapid acceleration or deceleration, impact, blast waves, or penetration by a projectile. Brain function is temporarily or permanently impaired and structural damage may or may not be detectable with current technology.

In some preferred embodiments, a drug regimen or electrical stimulus is used to treat brain trauma or neurological disease. In one embodiment, Transcranial Current Stimulation (TCS) is applied to a patient.

The TCS electrical stimulation may be applied using EEG electrodes or it can be delivered by other anode and cathode electrodes (i.e., anode sensors or cathode sensors placed from a different system) designated for TCS treatment (see for example Teskey et al., (2003) Neurol Res. December; 25(8):794-800; Utz et al., (2010) Neuropsycholegia. August; 48(10):2789-810). For example, sponges may be attached to graphite composite sensor pads sized for anode and/or cathode to ensure proper contact with the subject. The TCS device, in embodiments of the disclosed technology, directs anodal or cathodal non-invasive brain stimulation to one or more of the connected site locations on the subject. Stimulation can be delivered as transcranial current, or other effective current type, in amounts of about 0.1 mA to about 2 mA, about 0.2 mA to about 1.5 mA, about 0.25 mA to about 1 mA, about 0.1 mA to about 1 mA, about 1 mA to about 2 mA, or about 0.5 mA to about 1 mA. Other types of electrical stimulation that are known in the art can be used with the current invention.

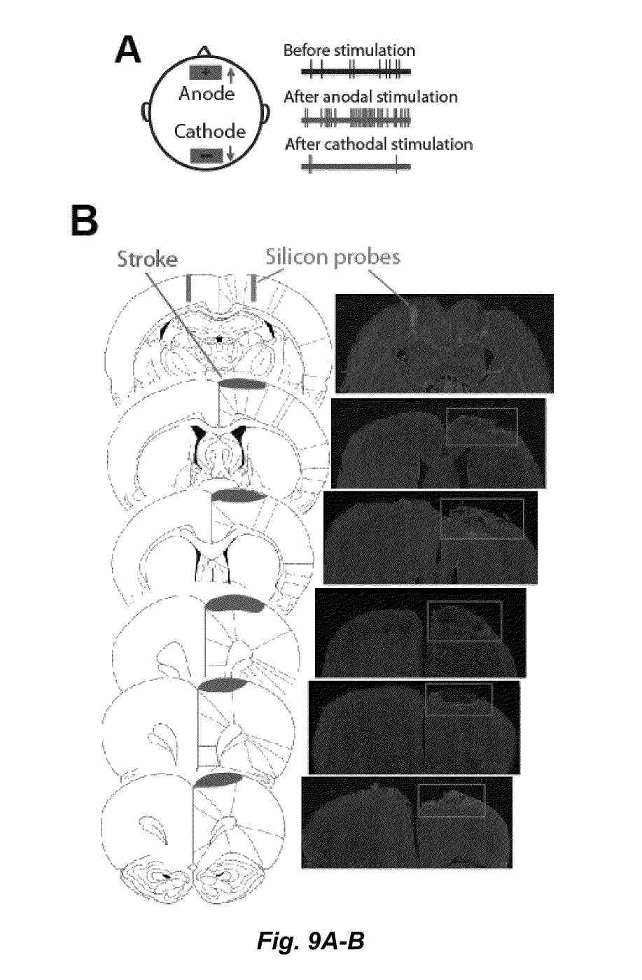

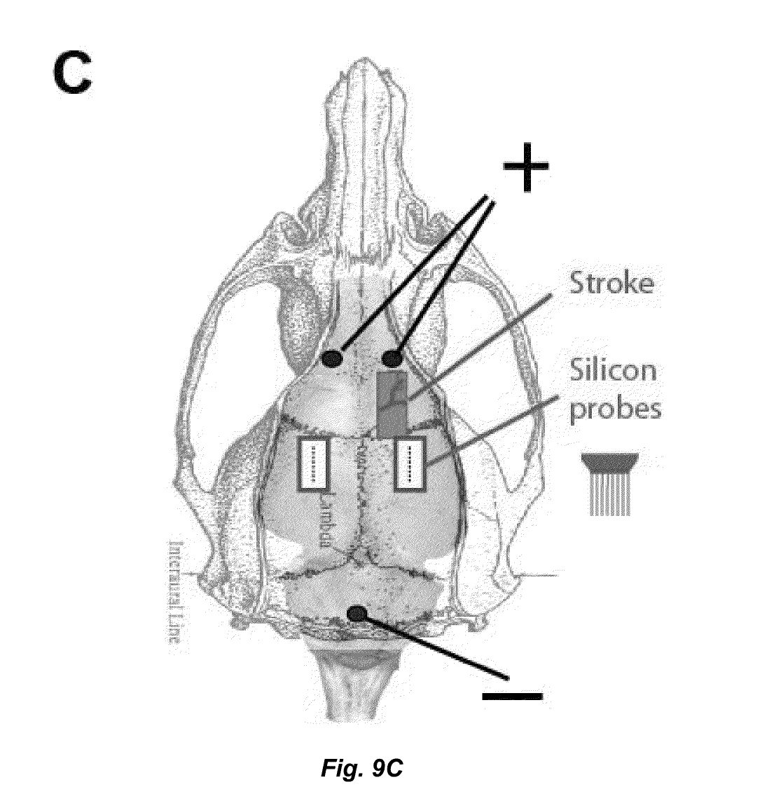

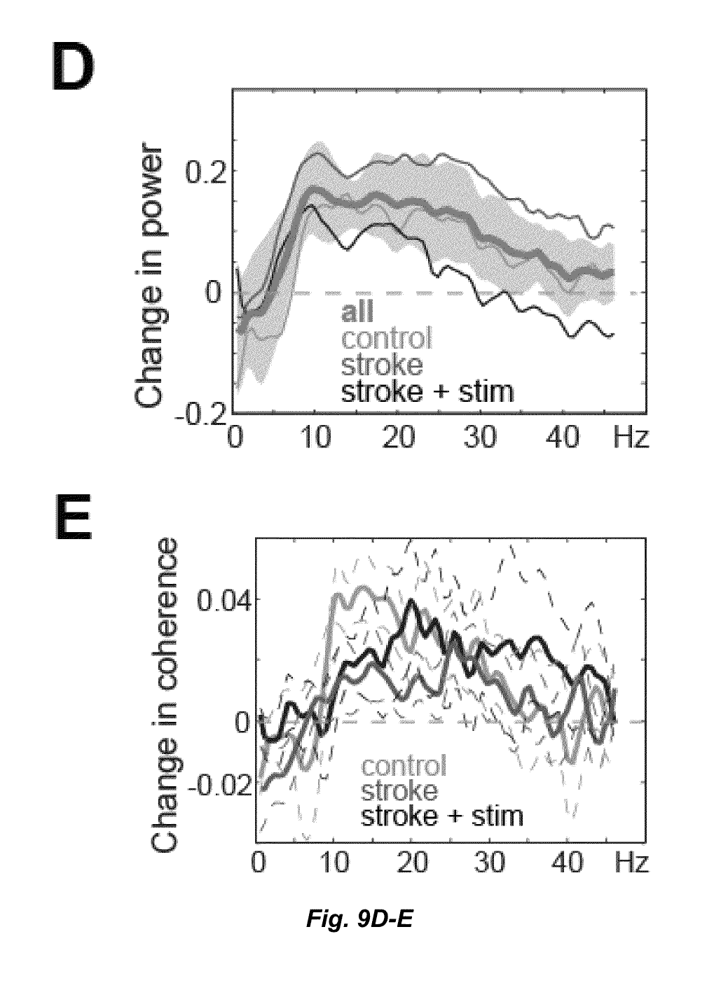

In one embodiment, at least one anode electrode is placed on or near the front of the frontal lobe. At least one cathode electrode can be placed on or near the rear of the frontal lobe. At least one recording electrode can be placed on or near the frontal lobe. An electrical stimulation can be applied through the electrodes. The brain biomarkers can be measured before, during and after electrical stimulation by the recoding electrode and sent to a recording device. The placement of electrodes in this configuration allows for control over the polarity of stimulation, and when it has an effect on neuronal stimulation (e.g., anodal stimulation has an opposite effect on neural spiking versus cathodal stimulation). Monitoring of brain biomarkers are then used to determine if the electrical stimulation has a positive effect on brain plasticity, or conversely, whether the stimulation has a negative effect on brain plasticity (see FIGS. 8, 9A-C).

In addition to electrical stimulation being used to treat brain trauma or neurological disease, it may be desirable to use a drug delivery system independently or in combination with electrical stimulation to result in the stimulation parameters of the present invention. Drug delivery may be used independent of or in combination with a lead/electrode to provide electrical stimulation and chemical stimulation. In some embodiments, when used, a drug delivery catheter can be implanted such that the proximal end of the catheter is coupled to a pump and a discharge portion for infusing a dosage of a pharmaceutical or drug. Implantation of the catheter can be achieved by combining data from a number of sources including CT, MRI, or conventional and/or magnetic resonance angiography into the stereotactic targeting model. Thus, implantation of the catheter can be achieved using similar techniques as discussed above for implantation of electrical leads, which is incorporated herein. The distal portion of the catheter can have multiple orifices to maximize delivery of the pharmaceutical while minimizing mechanical occlusion. The proximal portion of the catheter can be connected directly to a pump or via a metal, plastic, or other hollow connector, to an extending catheter. In other embodiments, the drug compounds are administered using well known means such as through an oral route or through hypodermic injection.

Herein, stimulating drugs comprise, but are not limited to medications, anaesthetic agents, synthetic or natural peptides or hormones, neurotransmitters, cytokines and other intracellular and intercellular chemical signals and messengers, other agents such as zinc and the like. In addition, certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug. In addition, stimulation of an area herein includes stimulation of cell bodies and axons in the area.

Similarly, excitatory neurotransmitter agonists (e.g., norepinephrine, epinephrine, glutamate, acetylcholine, serotonin, dopamine), agonists thereof, and agents that act to increase levels of an excitatory neurotransmitter(s) (e.g., edrophonium; Mestinon; trazodone; SSRIs (e.g., flouxetine, paroxetine, sertraline, citalopram and fluvoxamine); tricyclic antidepressants (e.g., imipramine, amitriptyline, doxepin, desipramine, trimipramine and nortriptyline), monoamine oxidase inhibitors (e.g., pheneizine, tranylcypromine, isocarboxasid)), generally have an excitatory effect on neural tissue, while inhibitory neurotransmitters (e.g., dopamine, glycine, and gamma-aminobutyric acid (GABA)), agonists thereof, and agents that act to increase levels of an inhibitory neurotransmitter(s) generally have an inhibitory effect (e.g., benzodiasepine (e.g., chlordiazepoxide, clonazepam, diazepam, lorazepam, oxazepam, prazepam alprazolam); flurazepam, temazepam, or triazolam). (Dopamine acts as an excitatory neurotransmitter in some locations and circumstances, and as an inhibitory neurotransmitter in other locations and circumstances.) However, antagonists of inhibitory neurotransmitters (e.g., bicuculline) and agents that act to decrease levels of an inhibitory neurotransmitter(s) have been demonstrated to excite neural tissue, leading to increased neural activity. Similarly, excitatory neurotransmitter antagonists (e.g., prazosin, and metoprolol) and agents that decrease levels of excitatory neurotransmitters may inhibit neural activity. Yet further, lithium salts, anesthetics (e.g., lidocane), and magnesium may also be used in combination with electrical stimulation.

Still further, the present invention can comprise a chemical stimulation system that comprises a system to control release of neurotransmitters (e.g., glutamate, acetylcholine, norepinephrine, epinephrine, and/or dopamine), chemicals (e.g., zinc, magnesium, or lithium) and/or pharmaceuticals that are known to alter the activity of neuronal tissue. In still other embodiments, currently known drugs that are effective in treating a disease can be administered to the patient, where the drug or drugs with the highest increase in electrical brain biomarkers (e.g., an increase in the brain desynchronized state) is continued as a treatment.

By monitoring a patient's electrical brain biomarkers in response to the drug treatment regimen, the user can vary the amount of dosage of the drug, length of time the drug is administered, the choice of drug or combination of drugs to better treat the disorder. For example, a patient can be administered a series of drugs and the user can detect a near real time increase or decrease in an electrical brain biomarker such as brain desynchronization. The administered drug having the highest increase in brain desynchronization is chosen for the drug treatment. The user can than vary the dosage or other variable and to achieve the best possible drug regimen based on the electrical brain biomarker readings. Stimulus can also include behavioral and/or cognitive therapy. Moreover, behavioral and cognitive therapies may be best applied to a patient when the patient is already in the desynchronized brain state. In some embodiments, the subject receives a combination of some or all of these therapies.

Also contemplated in the present invention is a method to test pharmaceutical compounds on patients, including both human and non-human patients. Non-human patients represent an ideal model for testing the efficacy of pharmaceutical compound on test subjects. For instance, a series of pharmaceutical compounds can be administered to a non-human patient. By measuring the electrical brain biomarkers, it can be determined which drugs have a more positive effect on the patient to bring about, for example, an increase in rehabilitation from a brain trauma. In one embodiment, the electrical brain biomarker that is measured in an increase in brain desynchronized brain state in response to a pharmaceutical compound.

In another embodiment, the efficacy of a pharmaceutical compound can be administered to determine the efficacy of promoting an increase in learning ability. In this instance, brain desynchronization is measured in response to the administered pharmaceutical compound. A pharmaceutical compound that causes an increase in brain desynchronization can also cause an increase in brain plasticity.

In some embodiments, the patient is a human. In other embodiments, the patient is a non-human mammal A non-human patient can be, but is not limited to, a farm animal (e.g., a cow, sheep, pig, and the like), a companion animal (such as a cat, dog, or horse, and the like), a non-human primate, or a rodent (such as a mouse or rat).

### General Apparatus Components

In one or more examples of the apparatus described herein, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques described herein can be fully implemented in one or more circuits or logic elements.

The techniques of this disclosure may be implemented in combination with a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

In some embodiments, the apparatus or more specifically, the processor is capable of determining in near-real time a brain plasticity index. The index can be calculated as the ratio between the gamma spectra power output and the delta spectra power output. In other embodiments, the brain plasticity index can be calculated as the power output in 2-5 Hz band divided by the power output in 35-53 Hz band and normalized (divided) by total power output in the 1-200 Hz band.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” refer to at least one and can include plural reference unless the context clearly dictates otherwise. Thus, for example, a reference to “a compound” includes at least one compound and can include a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual element, the composition, or the embodiment. The term about can also modify one or both end-points of a recited range as discuss above in this paragraph.

As will be understood by the skilled artisan, all numbers, including those expressing quantities of ingredients, properties such as molecular weight, treatment conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein the term “local field potential” or “LFPs” refer to a particular class of electrophysiological signals, which are related to the sum of all neuronal activity within a volume of tissue.

As used herein, “near-real time analysis” refers to analytics that are processed and analyzed as soon as they are measured, ranging from milliseconds to tens of seconds.

As used herein, “low frequency brain waves” refers to brain waves with a frequency of 10 Hz or less.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Sequential Neural Activity Patterns Evoked by Sensory Stimulation are Enhanced During Cortical Desynchronization

Example Summary.

Recently, Applicant have shown that brain plasticity could be linked to desynchronized (attentive-like) brain state regardless of method used to desynchronize cortical activity (Bermudez et al., (2013) Neuron 79, 555-566). Those findings suggest novel view on plasticity, namely that plasticity mostly depends on general state of neural network. Here, Applicant propose method and device based on the above idea to improve learning and brain recovery from neurological disorders.

Interestingly, it was shown that under general anesthesia that induces brain synchronization, it is possible to also induce periods of desynchronized brain activity using variety of different methods (see FIG. 6 for an example of neuronal spiking patters in synchronous and desynchronous brain states). For example, it can be achieved with tail pinch, infusion of carbachol in hypothalamus, or with IP injection of amphetamine to list just a few methods. Surpassingly, although each of the listed methods works by different mechanisms to induce desynchronization, in all cases it resulted in increased brain plasticity. While sensory evoked response in synchronized and desynchronized state are similar, only in desynchronized network state patterns of spontaneous activity following stimulation showed stimulus specific modification. Stimulus evoked sequential activity pattern, patterns of spontaneous activity preceding stimulation and following stimulation. Note that after stimulation spontaneous patterns are reorganized to resemble stimulus evoked pattern. Such reorganization is a gradual process, and spontaneous patterns become more similar to evoked patterns gradually as stimulation progresses. After stimulation stops spontaneous patterns continue to show stimulus specific similarity. Crucially, the extent of the plastic changes due to stimulation correlates with cortical state, where more desynchronized activity is correlated with more plasticity. Considering that in other studies, enhanced desynchronization of cortical activity has been linked to increased attention, those results are consistent with idea that Applicant learn better when Applicant pay more attention which desynchronize our brains more.

Results.

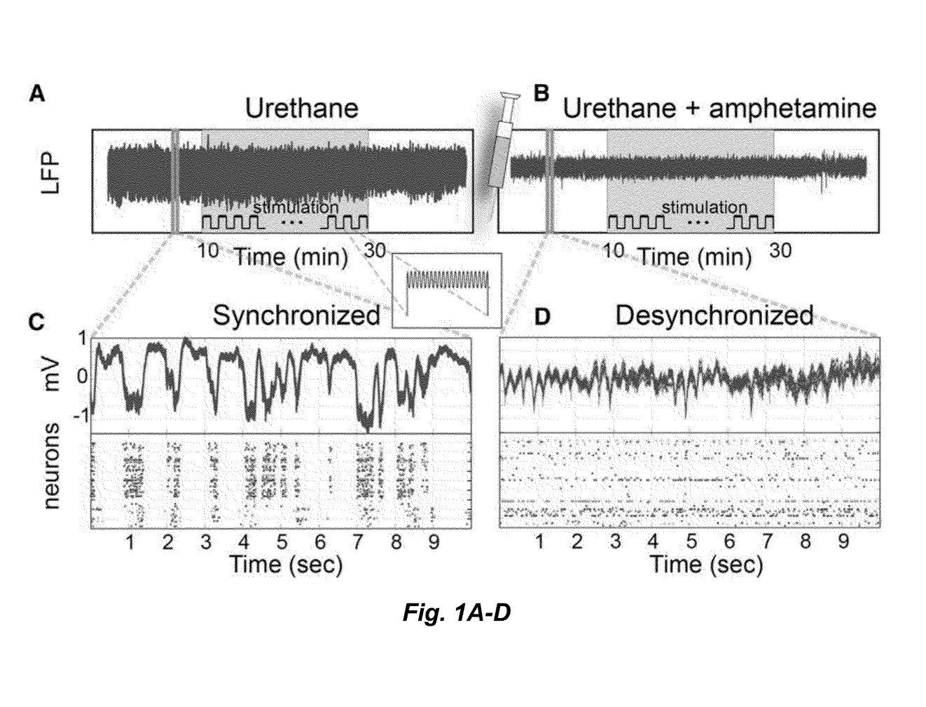

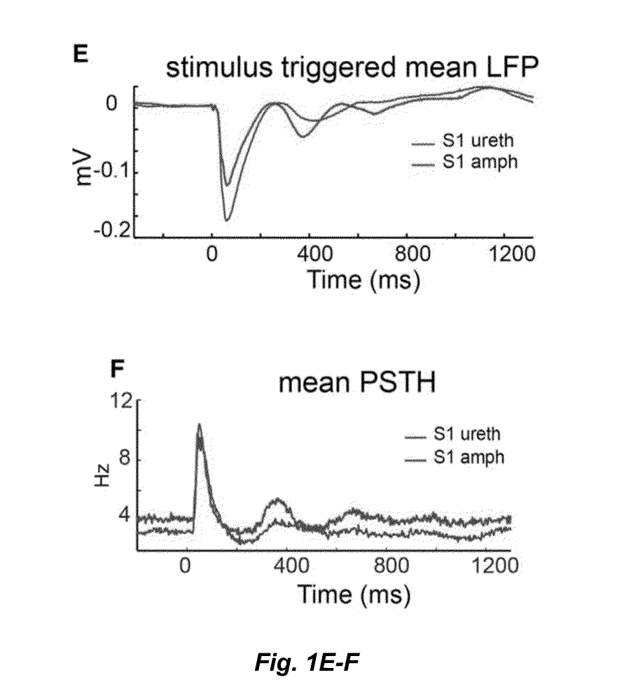

Applicant first investigated changes in spontaneous activity patterns induced by sensory stimulation by recording activity from neuronal populations in primary somatosensory cortex (S1). Under urethane anaesthesia (FIG. 1A), brain activity showed a synchronized state with characteristic slow wave oscillations in which generalized bursts of population activity (UP states) were interspersed with periods of neuronal silence (DOWN states) (FIG. 1C bottom). UP states were accompanied by negative deflections of the local field potential (LFP) (FIG. 1C top), indicative of synchronized synaptic inputs. Urethane promotes a condition of behavioural unconsciousness that closely mimics the full spectrum of natural sleep, although the duration of DOWN states is reported to be shorter in natural sleep as compared to anesthetized conditions. Injection of amphetamine rapidly changed the brain state; within few minutes after injection, cortical activity transitioned to a strongly desynchronized state, which lasted for at least 30 minutes (FIG. 1B, D). Tactile stimulation did not change either synchronized or desynchronized brain states (FIG. 1A, B—shaded area). Surprisingly, the average stimulus-triggered responses in S1 were very similar in synchronized and desynchronized states despite large differences in spontaneous neuronal activity among these states (FIG. 1E, F).

Spontaneous Sequential Activity Patterns are Modified by Tactile Stimulation.

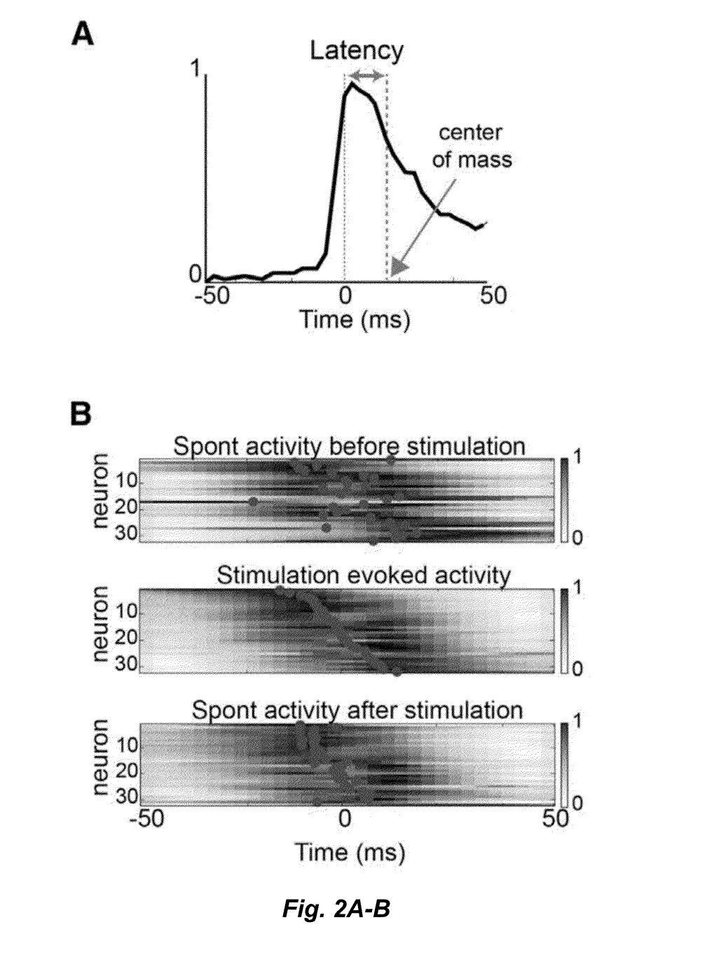

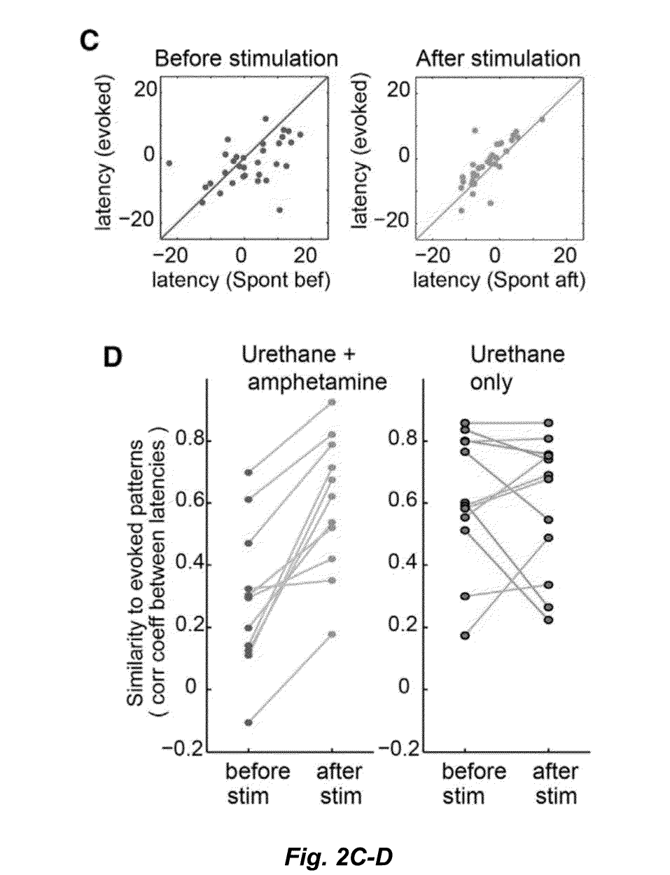

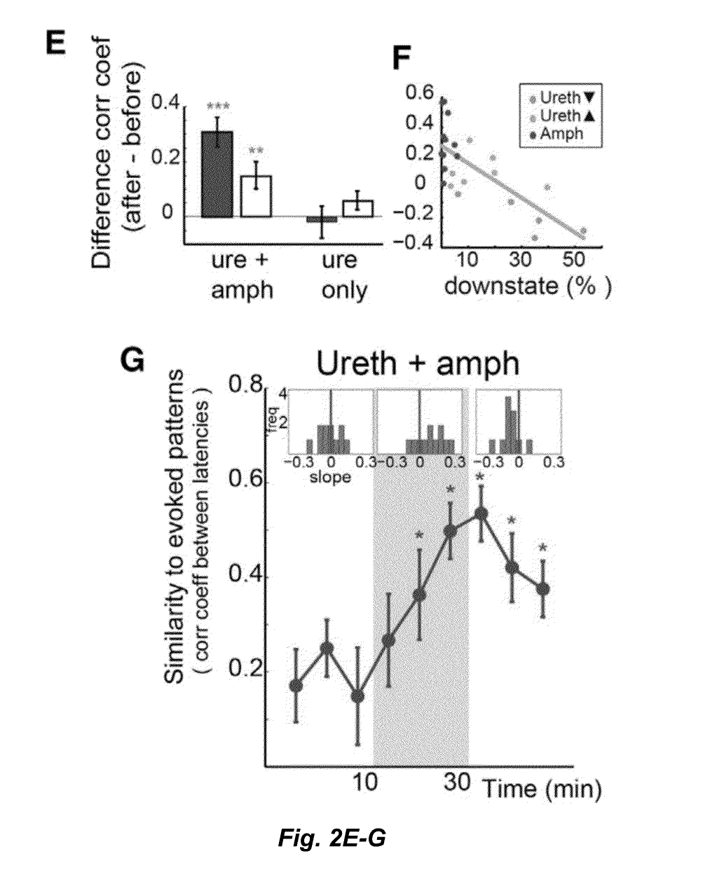

To investigate fine-scale temporal changes in spontaneous neuronal activity induced by sensory stimulation, Applicant first calculated the relative latency of each neuron. This reflects its timing in relation to other neurons based on cross-correlogram analysis (see Experimental Procedures below, and FIG. 2A). FIG. 2B shows cross-correlograms of 32 neurons from a representative experiment, sorted by latency during the stimulation period after amphetamine injection (middle panel). Consistent with previous results from auditory and visual cortex, neurons showed similar temporal patterns during spontaneous and stimulus-evoked conditions. For example, neurons that were firing earlier than other neurons during stimulation also tended to fire earlier than other neurons during spontaneous activity before or after tactile stimulation (FIG. 2B, top and bottom panel respectively). This is explicitly shown in FIG. 2C, where latencies from stimulation periods are compared to latencies from spontaneous periods for the same neurons. Note that latencies after stimulation are more similar to latencies during the stimulation period than to spontaneous latencies before stimulation (right and left panel in FIG. 2C respectively).

Applicant quantified this effect by comparing the correlation coefficient of latencies from stimulated and spontaneous periods. FIG. 2D shows such correlation coefficient values for all rats. Consistent with data presented in FIGS. 2B and 2C, the latency correlation increased significantly after stimulation for all animals under amphetamine (FIG. 2D left panel; FIG. 2E red bar: mean corr. coef. increase=0.31±0.062SEM, p=0.0001; t-test). For the animals without amphetamine injection (urethane only), the increase in latency correlation after tactile stimulation was not significant (FIG. 2D right panel; FIG. 2E blue bar: mean corr. coef. change=−0.03±0.06SEM, p=0.35; t-test). Similar results were obtained by computing latency from pairwise correlograms (FIG. 2E white bars; mean corr. coef. change: amph=0.098±0.023SEM; ureth=0.049±0.025 SEM; see Experimental Procedures). However, the rats in the urethane-only condition that do show an increase in latency correlation tended to have a more desynchronized brain state (FIG. 2F, corr. coef.=−0.66, p=0.01; see Supplemental Experimental Procedures below for definition of brain state measure). This indicates that in the desynchronized state induced by amphetamine or occurring spontaneously under urethane, the brain may be more plastic, such that the repeated tactile stimulation induced more extensive reorganization of spontaneous fine-scale temporal activity patterns. The increased similarity of evoked patterns and post-stimulation spontaneous patterns in this preparation could reflect similar processes as that underlying memory formation.

In order to investigate how spontaneous temporal patterns change over time, Applicant divided each experimental condition into 9 periods: 3 periods during the spontaneous activity before stimulation, 3 periods of the spontaneous activity occurring between the delivery of stimuli (e.g. the 1-second spontaneous activity intervals between the 1 second intervals of stimulation), and 3 periods for the spontaneous activity after stimulation (FIG. 2G). For each period, the latency correlation between spontaneous and evoked activity was calculated (during the 20 minute stimulation period, the stimulus was presented 600 times, and latency for evoked activity was calculated from all those 600 intervals of 1 second; to calculated for example, latencies from the first spontaneous period during stimulation, Applicant included data from the first 200 1 sec intervals between stimulation presentations).

In the amphetamine condition in 51, Applicant observed that, as expected, the latency correlation between pre-stimulus spontaneous and evoked activity did not change significantly (first three points in FIG. 2G; mean slope=−0.01±0.1 SD, p=0.73, t-test; FIG. 2G left inset). Following this, the latency correlation between spontaneous and evoked activity increases with time during stimulation (three points in the shaded area in FIG. 2G; mean slope=0.11±0.12 SD, p=0.01; middle inset). Once stimulation ceased, latency correlations decayed gradually (mean slope=−0.07±0.08 SD, p=0.01). Interestingly, this slow decrease in reactivation after stimulation is consistent with data from behaving animals, in which most reactivation is observed only within few minutes after tasks. To quantify the significance of sequence reverberation, Applicant compared averaged values of latency correlations before and after stimulation. The values of latency correlation were significantly higher after stimulation only for S1 in the amphetamine condition (p<0.0001, t-test), but were not significantly different for the urethane-only condition (p>0.1). Thus, in anesthetised rats injected with amphetamine that induced brain state desynchronization, sensory stimulation caused a gradual reorganization of spontaneous activity patterns in S1, and the ‘memory’ of that stimulation persisted in the following spontaneous activity patterns.

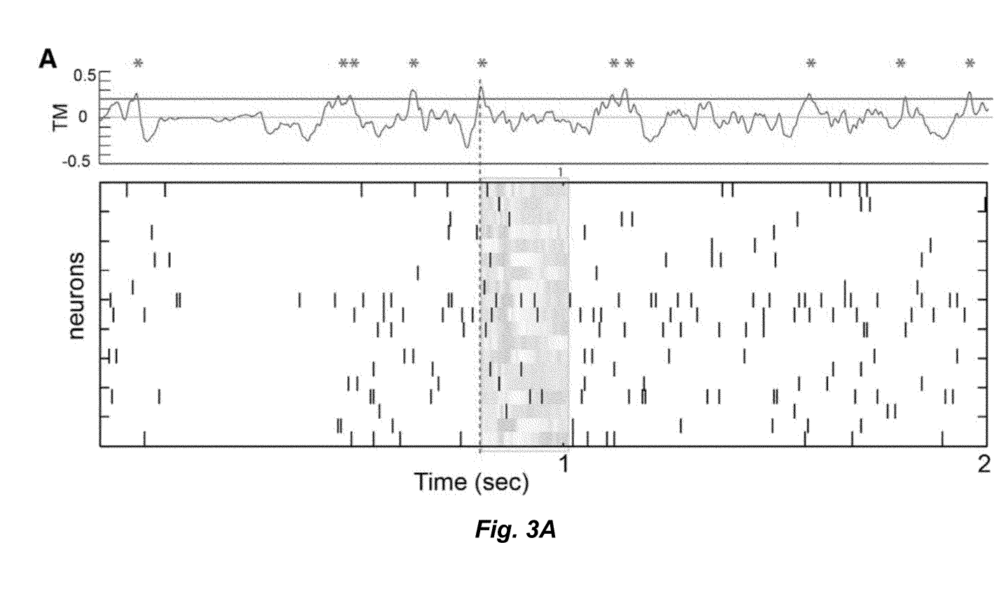

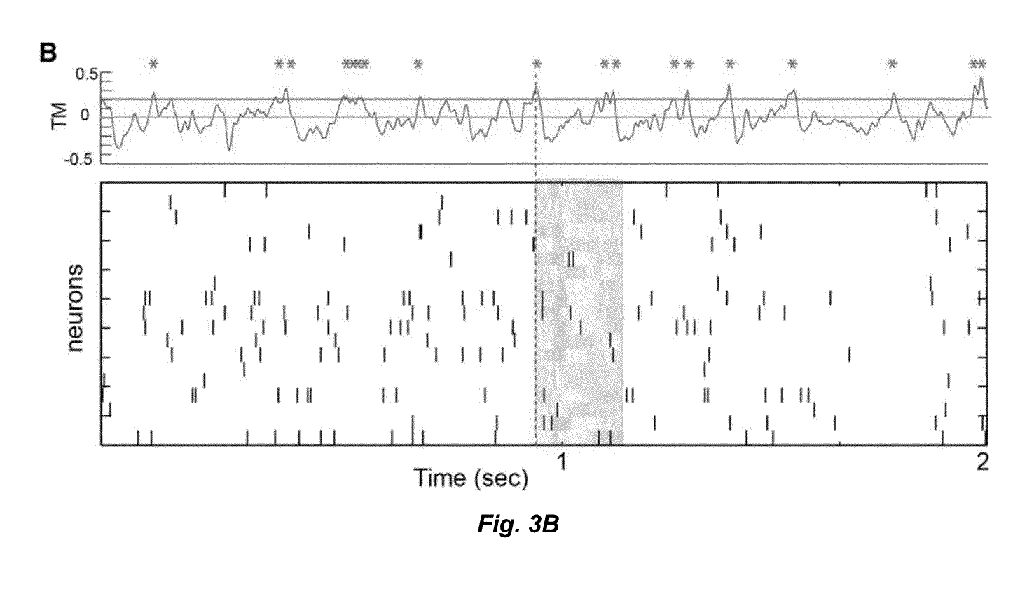

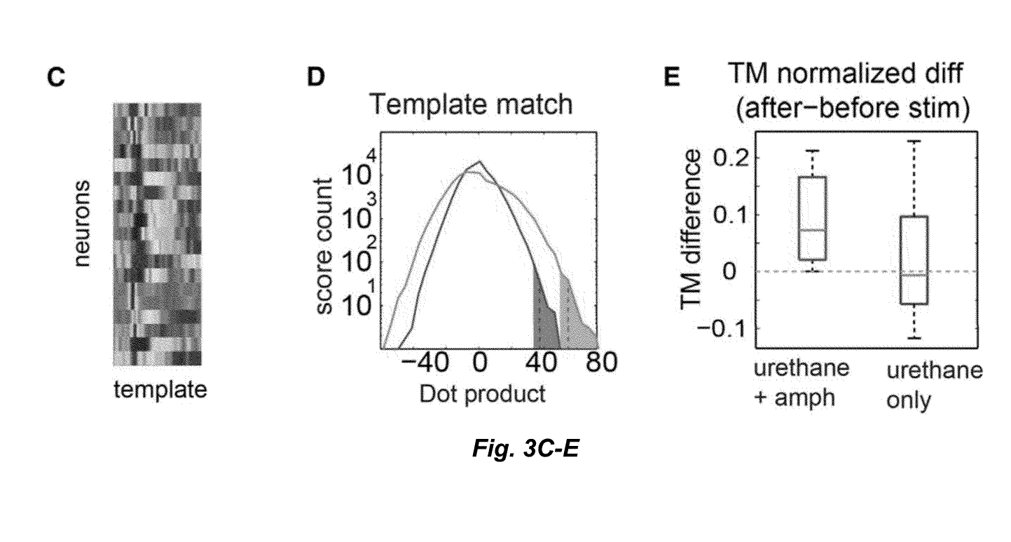

As an additional test that stimulus-evoked patterns in S1 are replayed during the following spontaneous activity, Applicant used template matching analysis as described in studies with behaving animals. Templates for each dataset consisted of average stimulus-triggered activity from 0-200 ms after stimulus onset. FIG. 3A-C shows template, sample raster plots and template matching scores for spontaneous activity before and after stimulation for a representative rat. Applicant found that in the amphetamine condition (but not the urethane only condition), the number of spontaneous patterns that closely matched the template was higher in the period following tactile stimulation (FIG. 3 D, E; pₐₘₚₜₕ=0.02, pᵤᵣₑₜₕ=0.52; t-test). As compared to the results obtained using the latency measure, reverberation disappeared faster after stimulation when analyzed with template matching. Although it is difficult to pinpoint the exact reason for this discrepancy, tests on simulated data indicate that latency measure could be more robust in small signal to noise regimes and less affected by any time compression of replayed patterns, thus giving better estimation of weak and varying reverbatory activity. Nevertheless, both analysis methods are otherwise consistent in revealing increased reverberation following stimulation in the desynchronized brain state (but not in the urethane-only condition).

Reactivation of Firing Rate Correlations.

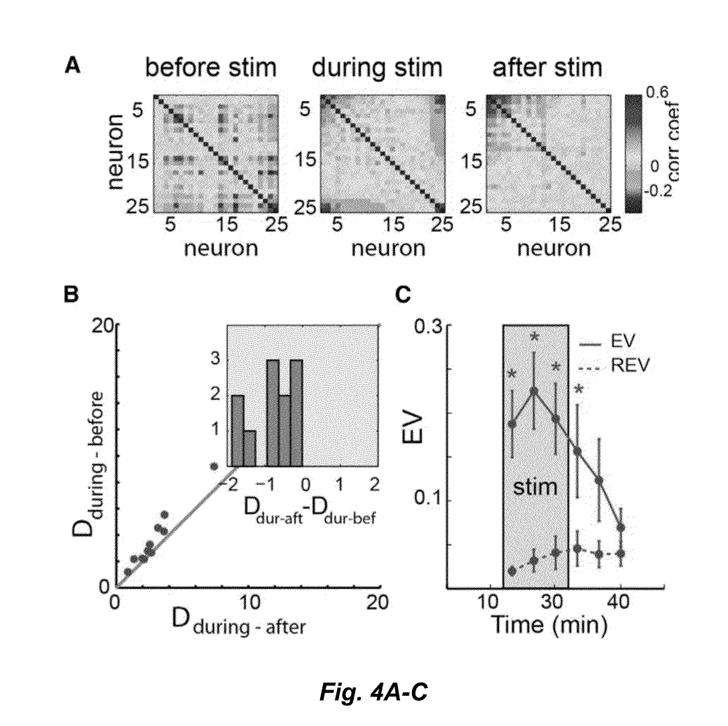

The foregoing analysis revealed that the timing relations among neurons during spontaneous activity have ‘memory’ of previous stimulus-evoked temporal patterns. However, given that the number of spikes fired by a particular neuron can be significantly affected by stimulus presentation, Applicant also investigated if firing rate correlations induced by tactile stimulation can be observed in subsequent spontaneous activity. To address this question Applicant smoothed spike trains with a Gaussian kernel (SD=130 ms), and calculated the correlation coefficient between all pairs of neurons. The resulting firing rate correlation matrices for units recorded in S1 for evoked and spontaneous periods during amphetamine are shown in FIG. 4A. Matrices for the spontaneous period after stimulation are more similar to matrices for the stimulation period than matrices for the spontaneous activity before stimulation (FIG. 4A).

In order to quantify similarities, Applicant calculated the Euclidian distance between the firing rate correlation matrices. For the amphetamine case, the distance between correlation matrices for evoked periods and the following spontaneous periods was smaller than the distance between correlation matrices for evoked and the preceding spontaneous periods, for all rats (FIG. 4B, p=0.003, paired t-test). However, in the urethane-only condition Applicant found a non-significant increase in similarity between correlation matrices for evoked and following spontaneous periods (S1: p=0.09 paired t-test). Using the correlation coefficient as an alternative measure of similarity between matrices resulted in similar findings. Our findings were preserved when the size of the smoothing kernel was varied from 30-180 ms. Thus, in the amphetamine case, the firing rate correlations induced by stimuli persist in subsequent spontaneous activity, which is consistent with memory reactivation studies in awake animals.

In order to quantify the temporal profile of firing rate replay, Applicant used the explained variance (EV) measure, which is a standard method applied to detect memory reactivation in behaving animal studies. EV is defined as the square of the partial correlation between firing rate correlation matrices during stimulation and subsequent activity, taking into account the correlations that existed prior to the stimulation. Similar to our analyses using latency correlations, evoked and spontaneous periods were subdivided into 3 smaller time sub-periods; the first spontaneous sub-periods were used as reference (PRE) for calculating EV on the following sub-periods (FIG. 4C). In the amphetamine condition, significant firing rate reactivation was observed during the stimulation period in 1 sec intervals of spontaneous activity in S1 (FIG. 4C, p<0.05, paired Kolmogorov-Smirnov-test). The reactivation slowly decreased after stimulation, similar to the decrease observed in the latency correlation analysis (compare FIG. 4C with FIG. 2G). Under urethane anaesthesia alone, Applicant also observed significant firing rate reactivation during stimulation periods, but these did not remain significant after stimulation.

Reverberation in Auditory Cortex.

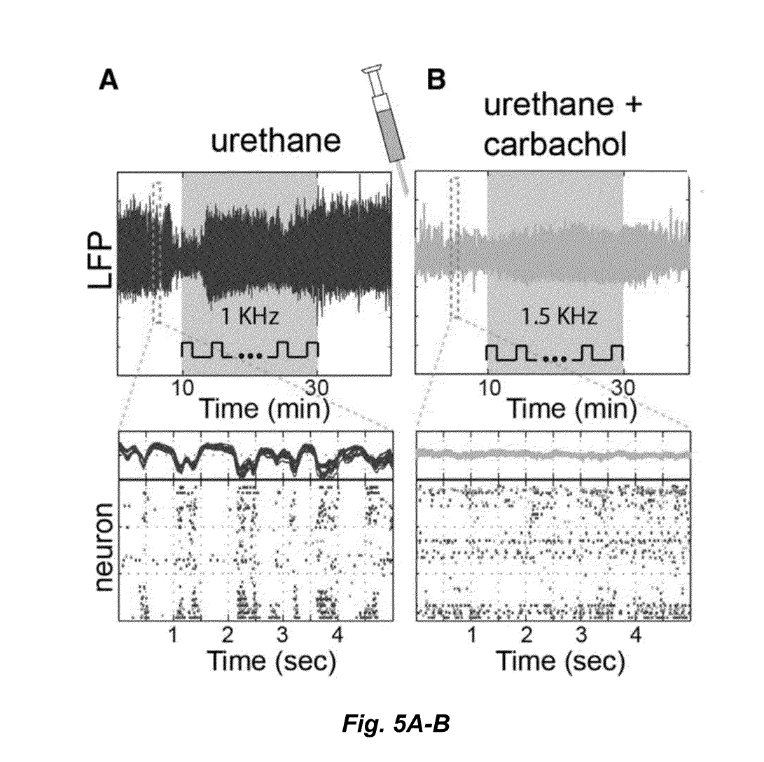

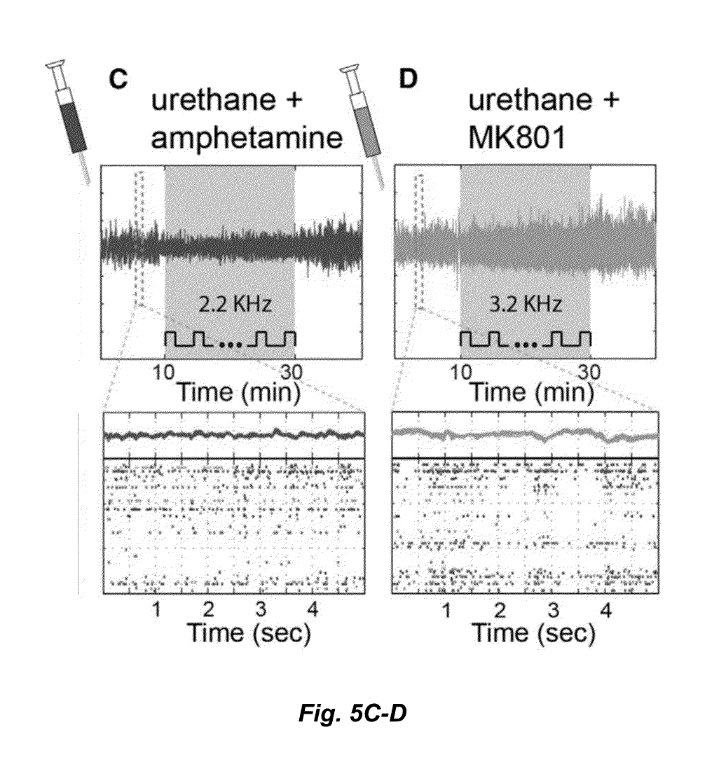

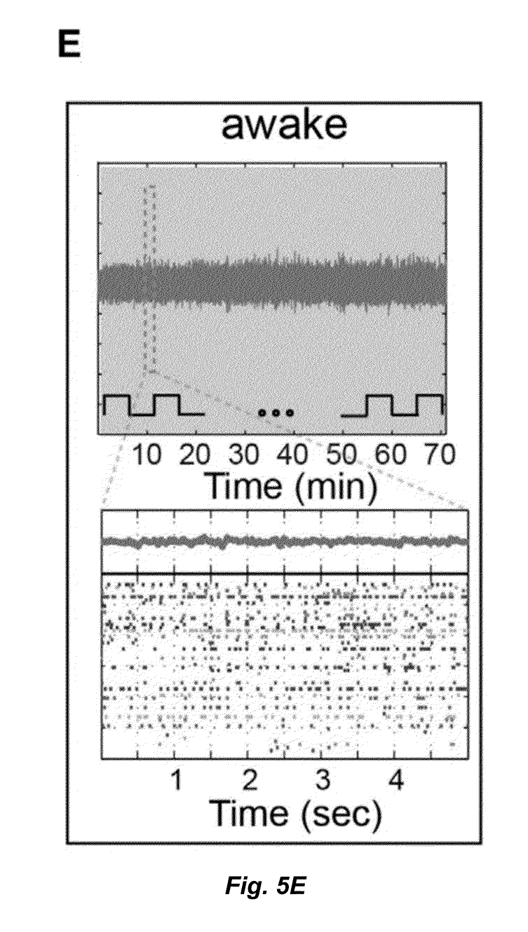

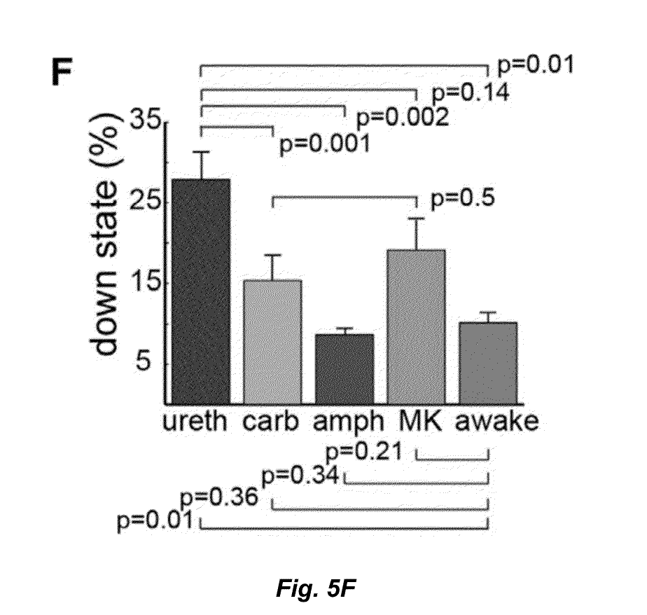

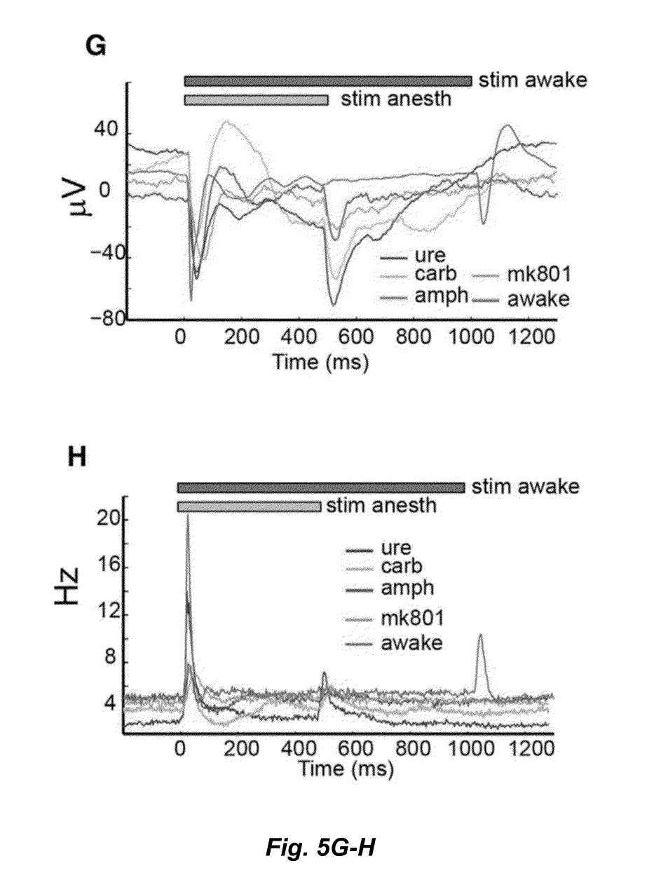

Applicant next sought to test whether the reactivation described above generalizes to other cortical systems and other mechanisms of desynchronization. Applicant therefore recorded in auditory cortex before, during and after presentation of tone stimuli, and induced desynchronization with amphetamine, tail pinch, or infusion of carbachol in the posterior hypothalamic nucleus (see Experimental Procedures below). The sequence of experimental conditions used to record population activity in A1 in urethane anesthetized rats is illustrated in FIG. 5A-D. In every experimental condition, Applicant recorded 10 min of spontaneous activity, followed by 20 min of auditory stimulation with pure tones, followed by 10 min of spontaneous activity (see Experimental Procedures below).

Under urethane anaesthesia, auditory cortex showed similar activity as in S1: large fluctuation of LFP associated with alternation between UP and DOWN states characteristic of the synchronized brain state (although short periods of spontaneously occurring desynchronized periods were also observed as reported before; FIG. 5A). Tail pinch or infusion of carbachol resulted in desynchronization of the brain state (FIG. 5B). Injection of amphetamine also induced desynchronization, but in this case desynchronization was more stable in time (FIG. 5C). In the last part of the experiment, each rat was injected with an NMDA receptor antagonist (MK801). After MK801 injection, the auditory cortex persisted in a desynchronized state, although more short periods of neuronal silence resembling DOWN states tended to occur toward the end of the experiment (FIG. 5D).

To directly compare results obtained in desynchronized brain state in anesthetized animals with processes occurring in awake rats, Applicant also analysed population activity recorded in auditory cortex in 3 awake, head-restrained rats (FIG. 5E). Applicant did not find significant differences between desynchronized brain states in awake and anesthetized animals based on analysis using the brain state index (FIG. 5F, the brain state index is defined as the percent of time that the neuronal activity spent in DOWN states as previously described). Furthermore, stimulus-triggered LFPs were similar for awake and anesthetized animals (FIG. 5G). For spiking activity, stimulus triggered onset and offset responses in anesthetized animals showed similar sharp increase and duration as in awake rats, although the amplitude of response was higher in awake animals (FIG. 5H). Altogether, these results suggest that cortical activity in the desynchronized state in anesthetized rats shows similar properties as in awake animals.

Discussion.

Memory formation is one of the most important processes in the brain, yet the neuronal dynamics underlying this process are only beginning to be understood, partly due to the technical difficulty of recording from large neuronal populations in behaving animals. Here, Applicant report that the hallmarks of memory formation and memory replay—stimulus-induced sequential activity patterns that reactivate spontaneously—can also be observed in urethane-anesthetised rats. In this preparation, population recordings and other brain manipulations can be more easily performed, thus providing a convenient model for electrophysiological study of mechanisms leading to formation of sequential patterns implicated in memory processes. Furthermore, Applicant found similar replay in both somatosensory and auditory cortices, suggesting this may be a general mechanism in the cortex. Although previous studies using voltage-sensitive dye imaging in anesthetised animals have shown that ongoing spontaneous activity can reflect stimulus-evoked spatial patterns on a coarse spatial scale, our findings provide a major refinement of these results by demonstrating replay of fine-scale sequential spiking patterns (FIG. 2 and FIG. 3) that is more analogous to sequential spiking patterns observed during memory replay in freely-moving animals.

In addition, our study indicates the importance of brain state during stimulus presentation. Although multiple studies show that most memory replay occurs during synchronized states, e.g. during slow wave sleep, the importance of the brain state during encoding is not clear. It is known that electrically evoked LTP is suppressed in this state, so there is a precedent for our current finding that presentation of stimuli during a desynchronized state, as compared to the synchronized state, is significantly more effective in inducing lasting reorganization of temporal patterns (FIG. 2), which subsequently results in stronger spontaneous replay of stimulus-induced patterns.

Applicant questioned why the induction of desynchronized states in anesthetised animals would facilitate the formation of tactile ‘memories’. A comprehensive explanation is lacking, but multiple lines of evidence suggest that desynchronization may be associated with increased brain plasticity. For example, amphetamine-induced desynchronization is also accompanied by increased extracellular levels of neuromodulators such as dopamine, which are implicated in the facilitation of memory consolidation in neocortex. Amphetamine also reduces extracellular GABA concentrations, and stimulates glutamate release. These mechanisms are believed to be responsible for enhanced cortical plasticity after amphetamine injection.

Amphetamine can also improve performance in tasks requiring attention, and attention is associated with enhanced desynchronization and enhanced representation of salient stimuli. Similarly, desynchronization induced by tail pinch and carbachol infusion in to the posterior hypothalamus involves activation of the cholinergic system, which is known to modulate diverse plastic processes in the hippocampus and neocortex. Multiple studies also show that acetylcholine enhances plasticity during presentation of specific sensory stimuli, allowing those specific sensory stimuli to evoke stronger or more prominent neuronal response. Thus, Applicant suggest that the brain is more plastic in the desynchronized (attentive-like) state, which may result in better ‘encoding’ of tactile stimuli that, in turn, results in stronger reverberation during subsequent spontaneous activity. It remains to be determined if increased attention in the awake state could have an analogous enhancement of stimulus-evoked neural reorganization.

Applicant also investigated what plasticity mechanisms may be involved in replay activity, and Applicant found that it was suppressed by application of an NMDA receptor antagonist. Those results are in line with studies showing that the consolidation of recent information into long-lasting memories appears to depend on NMDA function both during and shortly after an experience. For instance, localized interference of NMDA receptor function after an experience impairs recall tested many hours or days later, as has been shown in a number of brain structures including hippocampus, auditory cortex, and prefrontal cortex. NMDA receptor antagonism also blocks experience-dependent expansion of hippocampal ‘place fields’. Further, NMDA receptors play a crucial role in the modification of neural connectivity during or following experiences. NMDA antagonists attenuate experience-driven reorganization of the body map in S1 of awake animals, and retard value-related changes of neural firing in orbitofrontal cortex of behaving animals. These data indicate that neural reactivation causes formation of long-term memories via NMDA-dependent changes in synaptic strength. The pattern reactivation phenomena Applicant describe here is also dependent on NMDA receptors and is therefore consistent with the mechanisms of memory consolidation in the awake state.

Previous studies have suggested that ‘reverberating’ patterns are similar to spontaneous patterns that precede specific sensory experience. This phenomenon is termed “preplay.” Similarly, in the pre-task spiking patterns in medial prefrontal cortex have obvious similarity to patterns during the task and patterns replayed after the task. The data presented here are consistent with these results, and suggest that repeated stimulation induces only gradual changes to existing spiking patterns. For that, the relationship between stimulus-evoked (or reverberating) sequences to prior patterns occurring spontaneously is a very important question. Applicant have previously shown that stereotypical patterns of population activity are associated mostly with the beginning of UP states and that stimulus-evoked patterns have strikingly similar temporal structure to such spontaneous patterns. Furthermore, even in desynchronized brain states, population activity is composed of bursts of population activity with similar temporal structure to patterns during UP states in synchronized states.

Similar sequential patterns with stereotyped spatiotemporal dynamics have been also observed in vitro, indicating that network UP states could be circuit attractors. Together these in vitro and in vivo studies suggest that connectivity patterns at the local level impose significant constraints on activity propagation, thus leading to formation of similar sequential population patterns both spontaneously and during stimulation (although different stimuli produce slightly different variations of that sequential pattern. The results presented here are consistent with these ideas, and Applicant suggest that repeated stimulation may induce stimulus-specific changes in the underlying neuronal connectivity, especially when stimuli are presented in desynchronized brain states. Applicant speculate that these neuroanatomical changes could be the reason why spontaneous activity, which propagates through the same cortical circuits as evoked activity, becomes more similar to previously presented evoked patterns.

Applicant also speculate that the reverbatory activity described here may relate to memory formation in behaving animals. Although the mechanisms underlying memory formation processes are still not well understood, there is a body of theoretical work that predicts reverberation and/or reactivation as fundamental components of memory consolidation. Such phenomena have since been observed in the hippocampus and cortex of behaving animals. These observations, like ours, are consistent with the theory, but do not demonstrate that memory depends on this replay. However, more recent evidence suggests a direct link between replay and memory.

In hippocampus, the reverberation (reactivation) is associated with sharp wave ripple (SPWR) events, and studies have now shown that memory is impaired when SPWR are disrupted immediately following training. Furthermore, there are individual differences in reactivation and memory performance, and these are correlated. These data suggest that the replay of task-related activity is involved in memory processes. Note also that our experiments follow the same general design as ‘classic’ reactivation experiments. Applicant have a control period before an experience, a repetitive experience, followed by a test period. Applicant show that the activity in the test period resembles the activity in the repetitive experience after controlling for any preexisting similarity. The only difference is that the animal is not actually behaving but rather under anesthesia. By the fundamental definition of memory as a recapitulation of neural activity evoked by an experience, this is memory. Thus Applicant suggest that replay of stimulus-evoked patterns observed in desynchronized brain states in urethane-anesthetized rats, could be a useful model for studying mechanisms of memory.

### Experimental Procedures

Surgery and Recording.

Applicant used surgery and recording procedures that have been previously described in detail (Luczak et al., (2007) Proc. Natl. Acad. Sci. U.S.A. 104, 347-352.) Briefly, for somatosensory experiments, 11 Long Evans rats (400-900 g) were anesthetised with urethane (1.5 g/kg IP). Rats were then placed in a stereotaxic frame, and a window in the skull was prepared over primary somatosensory cortex (S1) hindlimb area (AP −1 mm; ML 2 mm; DV 1.5 mm) For auditory experiments, 8 Long Evans rats (250-350 g) were anesthetised with urethane (1.5 g/kg IP) and placed in a nasal restraint that left the ears free. A window in the skull (2×3 mm) was prepared over the primary auditory cortex. For all recordings, Applicant used silicon probes consisting of eight shanks (200 μm shank separation); each shank had 4 recording sites in a tetrode configuration (20 μm separation between sites, 160 μm² site area; 1-3 MOhm impedance; NeuroNexus Technologies).

The locations of the recording sites were determined to be layer 5 in S1 and in A1 based on histological reconstruction of the electrode tracks, electrode depth, and firing patterns. Desynchronization of brain state in the urethane auditory experiments was induced by applying: (1) 30 s to 1 min of pressure to the base of the tail (tail pinch, n=2) repeated 5-10 times in a 40 min period, or (2) by the application of 2 μL of carbachol (10 μg/μL, n=6) at a rate of 0.5 μL/min infused through a guide cannula (30 gauge) implanted into the right posterior hypothalamic nucleus (PH). Every 5-10 min over 40 min of that experimental condition, an additional 1 μL of carbachol was infused to prevent reoccurrence of synchronized brain state (Marguet et al., (2011) J. Neurosci. 31, 6414-6420).

After tail pinch or carbachol activation, animals were injected with amphetamine (1 mg/kg d-Methamphetamine HCl (MA; Sigma) dissolved in the sterile saline at a concentration of 10 mg/3 mL, IP), and after waiting 20 min for the effect of amphetamine to stabilize, Applicant recorded 40 min of neuronal activity. Then rats were injected with an NMDA antagonist (MK801, 0.1 mg/kg i.p.), and after waiting 20-30 min for drug effects to stabilize, Applicant again recorded for 40 minutes. During each experimental condition, Applicant recorded 10 min of spontaneous activity, followed by 20 min of stimulation, followed by 10 min of spontaneous activity (see details in sections below and in FIGS. 1 and 5).

The experimental procedures for the awake, head-fixed experiment have been previously described. Briefly, a headpost was implanted on the skull of the animal under ketamine-xylazine anaesthesia, and a crainiotomy was performed above the auditory cortex and covered with wax and dental acrylic. After recovery the animal was trained for 6-8 days to remain motionless in the restraining apparatus. On the day of the surgery, the animal was briefly anesthetized with isoflurane, the dura was resected, and after recovery period, recording began. Only experiments where the animal stayed motionless for at least one hour, indicated by stable, clusterable units, were included in this study (3/7 rats).

Tactile Stimulation.

The time course of the experimental protocol is illustrated in FIG. 1A and FIG. 1B. It consisted of two 40-minute periods; in the first period, the rat was only under urethane anaesthesia, in the second period the animal was additionally injected with amphetamine (1 mg/kg). Each recording period consisted of 10 min of spontaneous activity, followed by 20 minutes of tactile stimulation, and then another 10 min of spontaneous activity. The tactile stimulation consisted of 600 repetitions of 1 second stimulation at 20 Hz followed by 1 second without stimulation. The tactile stimulator consisted of a plastic rod attached at one end to a membrane of a speaker controlled by a computer. The other end of the rod was placed in contact with left hind limb.

Auditory Stimulation.

For auditory stimulation in anesthetized animals, the time course of experimental protocol was similar to that for tactile experiments in S1, and it is illustrated in FIG. 5 A-D. After 10 min of recording spontaneous activity, tones were presented for 0.5 s, interspersed with is of silence. This timing allowed for more off-to-on transitions of tones, which evoked the greatest response, than would be possible with the same period using tones of 1 s duration. Thus, 800 repetitions of tone stimuli were presented in the 20-minute stimulation period. For each experimental condition, Applicant used a different tone frequency during stimulation (urethane only: 1 kHz; tail pinch or carbachol: 1.5 kHz; amphetamine: 2.2 kHz; MK801: 3.2 kHz). For experiments with awake, head-restrained rats, auditory stimulation was presented for over 40 min in each animal. The pattern of stimulation consisted of repetitions of tones for one second followed by one second of silence. Activity occurring 200 ms after stimulus offset and before the next stimulus onset was regarded as spontaneous. Stimuli consisted of pure tones tapered at the beginning and the end with a 5 ms cosine window. In datasets from awake animals, Applicant did not have extended spontaneous periods proceeding or following stimulation period. Experiments took place in a single-walled sound isolation chamber (IAC, Bronx, N.Y.) with tones presented free-field (RP2/ES1, Tucker-Davis, Alachua, Fla.).

Latency.

In order to quantify temporal relations among neurons, Applicant calculated the mean spike latency as described previously. Briefly, for each neuron, latency is defined as the center of mass of a cross-correlogram of that neuron with the summed activity of all other simultaneously recorded cells (MUA), within a time window of 100 ms (FIG. 2A). Before calculating the center of mass, cross-correlograms were smoothed with a Gaussian kernel with SD=5 ms and normalized between 0 and 1 to discard effects of baseline activity. Thus this measure estimates the time when the corresponding neuron is most likely to fire with respect to the population activity. In addition to analysis of cross-correlograms between single neurons and multiunit activity as described above, Applicant also calculated latency from pair-wise cross-correlograms to look at temporal relations between neurons in more detail (FIG. 2E white bars). Applicant also confirmed latency measure stability over time.

Electrophysiological Recordings.

Extracellular signals were high-pass filtered (0.1 Hz) and amplified (1,000 times) using a 64-channel amplifier (Neuralynx). Signals were then digitized at 32 kHz. Units were identified and isolated by a semiautomatic cluster cutting algorithm (available at: klustakwik.sourceforge.net), followed by manual clustering (MClust). Multiunit activity that included clusters with low separation quality (isolation distance<20; Schmitzer-Torbert et al., (2005), Neuroscience 131:1-11) or firing rates lower than 0.03 Hz) were excluded from analysis. In the urethane only condition in 51, an average of 53 cells per rat passed this criterion for further analysis (24.1 SD, [17-102] range), while in urethane+amphetamine condition in 51, an average of 34 cells per rat passed criterion (18.7 SD, [9-71] range). Prior to recordings, 6 out of 11 rats received injections of amphetamine (1 mg/kg, i.p.) once a day for 14 days, with the last injection administered 3-6 weeks before recordings.

Because results from those rats were not different from control animals, Applicant combined data from all rats. For recordings in auditory cortex in anesthetized rats, 2 silicon probes (8-shank each) were inserted closely to each other in A1, yielding recording from 83±63SD well-isolated units on average with majority of cells being active across all experimental conditions. In the awake, head-fixed rats electrodes were estimated to be in deep layers of primary auditory cortex (Luczak et al., (2009), Neuron 62, 413-425). Only well-isolated neurons with firing rates higher than 0.03 Hz were used in this analysis. An average of 44 cells/rat passed this criterion for further analysis (9.53 SD, [38-55] range).

Brain State Measure.

In order to quantify the brain state, Applicant calculated the amount of time that the neuronal activity spent in DOWN states by smoothing the multi-unit activity with a Gaussian kernel (SD=40 ms) and computed the percent of time the activity was below 0.01 spike per 1 millisecond bin. To ensure that our results are not affected by differences in firing rates among animals, Applicant down sampled the multi-unit activity of all animals to match the number of spikes in the sample with the lowest firing rate. Applicant also checked that results were consistent if the SD of the smoothing kernel was varied between 16-128 ms. Applicant additionally used an alternative brain state measure based on the coefficient of variation of spiking activity, which yielded qualitatively similar results. Moreover, this brain state measure was also applied in and showed good agreement with LFP signatures of different brain states, i.e. power of LFP across frequency bands.

Template Matching.

Applicant used a template matching analysis to explicitly test the replay of stimulus-evoked patterns. This method consists of comparing the similarity between the average evoked-activity pattern (template) and the spontaneous activity. To calculate if the number of good template matches increased after stimulation, Applicant calculated the distributions of the matching values between the template and spontaneous activity before and after stimulation (FIG. 3D). From the distributions Applicant calculated the normalized difference of the average of the 0.1% of highest template matching values (V) after stimulation and before stimulation (the shaded regions in FIG. 3D are the 0.1% of highest matching values and the dotted lines correspond to their averages) for all rats as:

$$V = \frac{{M}_{0.1 \% aft} - {M}_{0.1 \% bef}}{{M}_{0.1 \% aft} + {M}_{0.1 \% bef}}$$

where M_{0.1% aft}(M_{0.1% bef}) is the average of the 0.1 percent of the largest matching values after (before) stimulation. Although template matching is frequently used to study memory replay (Euston et al., 2007; Tatsuno et al., 2006), Applicant found that the latency measure is a more robust estimate of stimulus induced changes in temporal activity patterns. For this reason, most of our reported analyses are based on latencies.

In order to build the template, Applicant z-score normalized the averaged neuronal activity within the first 200 ms after stimulus onset and smoothed it with a low-pass Gaussian filter (SD=6 ms; changing SD±4 ms did not affect our findings). For this analysis Applicant only included neurons with a firing rate above 0.6 Hz. The matching (M) between the template (T) and the spontaneous activity window (which was also z-score normalized) was calculated as the sum of the dot product of the template and an equal-sized sliding window (w) of activity across the analysis period: M=ΣᵢⁿT·wᵢ, where (•) is the dot product operator and n is the number of overlapping windows in the corresponding spontaneous activity period being analyzed. In addition, Applicant also used the following normalization for the template matching measure:

$${Σ}_{i}^{n} \frac{T \cdot {w}_{i}}{‖ T ‖ ‖ {w}_{i} ‖} ,$$

where ∥ . . . ∥ denotes the 2-norm of the spikes vector representation of the template or matched window. Using this normalization resulted in qualitatively similar results as using z-score normalization described above.

Explained Variance Analysis.

In order to quantify memory-trace reactivation over time, Applicant calculated the explained variance (EV) as previously described. Briefly, EV is the square of the partial correlation between firing rate correlation matrices during stimulation (STIM) and subsequent activity (POST), taking into account the correlations that existed prior to the stimulation (PRE). Applicant divided the recorded neuronal activity into 9 segments: 3 for the initial spontaneous activity (PRE), 3 for the stimulation period (each 6.6 min long), and 3 for the subsequent spontaneous activity. The stimulation period (STIM) was defined as the activity during each stimulation trial. POST periods consisted of spontaneous activity during inter stimulus intervals (1 sec intervals following each trial), and activity after the stimulation period.

To evaluate the significance of EV values Applicant used reversed EV as described. Reversed EV is calculated the same way as normal EV but PRE and POST periods are switched. To calculate firing rate correlation matrices, Applicant smoothed activity of each neuron with Gaussian kernel (SD=130 ms) and calculated the correlation coefficient between all pairs of neurons in the given period as described in. In our analyses Applicant did not exclude cell pairs belonging to the same tetrode, however repeating our analyses using such exclusion gave consistent results with our original analyses.

### Example 2

### Direct Current Stimulation Improves Limb Use after Stroke by Desynchronizing the Brain State

Example Summary.

Applicant collected multiple sets of preliminary data to more directly investigate relation between brain synchrony and stroke recovery (plasticity). For those experiments Applicant have chosen to use transcranial direct current stimulation (tDCS) method which was shown to improve stroke recovery and which can directly affect neuronal excitability. Briefly, with tDCS a constant, low current is applied directly to the brain using electrodes placed on the surface of the scalp above areas of interest. Effects of stimulations depend on polarity of electrode: anodal stimulation increases firing rate and the excitability of cortical neurons, and cathodal stimulation has opposite effect. The mechanisms by which tDCS affects brain are not fully understood but number of possible mechanisms including local changes in ionic concentrations, alterations in transmembrane proteins, and electrolysis-related changes in hydrogen ion concentration are implicated. tDCS can also have long term effects on the brain cytoarchitecture, for example, data from our lab showed increased neural density in stimulated cortical areas.

In first set of experiments animals received stroke in fore-limb area of motor cortex. During the same surgery electrodes were implanted to deliver tDCS during rehabilitation process. After 3 weeks of rehabilitation and behavioral testing (single pellet reaching task+tDCS) rats were anesthetized and silicon probe inserted in both hemispheres in somatosensory cortex in close proximity to stroke area. During acute recordings with silicone probes Applicant found that application of tDCS (through the same electrodes as chronic stimulation) increased LFP power in high frequencies and decrease in power in low frequencies (<5 Hz), which are the main characteristics of increased brain desynchronization. Thus those results indicated that beneficial effects of tDCS could be due to increased cortical desynchronization, which is consistent with our hypothesis that desynchronization increases plasticity and recovery.

Interestingly, Applicant also found that LFP coherence between hemispheres could be a novel and accurate marker for brain synchrony and stroke recovery. First, LFP coherence closely echoed changes in LFP power during brain desynchronization. Second, higher spontaneous coherence between hemispheres was correlated with better performance on reaching task during the last week before recordings. These are very important findings. First, it indicates that brain desynchronization may also facilitate communication between brain regions (increased coherence in high freq. bands), and second, coherence could provide electrophysiological measure (signature) of brain ‘healthiness’, as it is know that different neurological disorders (including stroke) is manifested by dysfunction of communication (reduction in functional connectivity) between brain regions.

### Materials and Methods

Skilled Reaching Task.

Fifteen male Long-Evans hooded rats received from Charles River (Ontario, Canada), aged 70-80 days (400 g-500 g), and were habituated to the experimenter for seven days. After habituation all rats were trained in the skilled reaching task, which is a reach-to-eat task designed to quantify and qualify skilled forelimb use. The skilled reaching apparatus was a rectangular Plexiglas box 40 cm long×45 cm high×13 cm wide with a 1 cm slot at one end and a shelf fixed to the outside of the box which was accessible through the slot (FIG. 11A). The shelf had two symmetric indentations that were 1.5 mm deep, 1.5 cm from the slot, parallel to each edge of the slot to provide stability and a consistent position for the food pellets to be placed. Small 45 mg food pellets were used (Bioserve, USA; Product # F0021).

Once trained to asymptote success levels with their preferred paw, rats received 20 pellets per day for five consecutive days in the reaching task to establish baseline reaching success values. In each trial, animals were required to walk to the rear end of the box prior to reaching for a pellet to readjust their body position. A successful reach was defined as an animal reaching for the pellet, grasping it and placing it in its mouth on the first attempt. Reaching Success (RS) for each rat and each session were calculated using the following formula: RS=number of successful reaches/20×100%. Data was scored by group-blind observers from recorded videos.

Before inducing lesions and implanting stimulating electrodes (see next section) animals were assigned to experimental groups to match paw preference and reaching success between groups. Six days following surgery animals were tested in skilled reaching for three weeks and after a four-week break were re-tested for four consecutive days. During retesting animals showed stable performance consistent with pre-break performance.

Photothrombosis and Electrode Implantation.

Focal photothrombosis was induced in the forelimb area of primary motor cortex contralateral to the forelimb preferred in the reaching task. Animals were anesthetized using 4% isoflurane in a mixture of 1.5% oxygen and secured in a stereotaxic frame (David Kopf, Germany). The skull over the motor cortex was thinned using a fine dental burr in a rectangular shape from Bregma −1.0 to 4.0 anterior/posterior and Bregma 1.0 to 4.0 medial/lateral. A cold light source (Schott KL 1500, Germany) with an aperture of same size and shape as the partial craniotomy was positioned over the skull. The skull was illuminated at maximum light settings for 20 minutes.

During the first 2 minutes of illumination Bengal Rose dye solution was injected through a tail vein (20 mg/kg, 10% solution in 0.9% saline). When the illumination period was completed, animals were implanted with stainless steel screws used for electrical stimulation (1 mm diameter; Small Parts, USA). Pilot holes were drilled in the skull bilaterally at coordinates: +4.5 A/P, ±1.0 M\L (FIG. 9A-B). A dental acrylic skullcap was fashioned on the exposed skull, engulfing the electrodes, connecting wires, and connecting plug (Ginder Scientific, ON, Canada). Once the acrylic was hardened, animals were removed from the stereotaxic frame, placed on a heating pad and monitored until they were awake (Paxinos et al., (1998) 4th Edition ed. Academic Press: San Diego).

Lesion Analysis.

DAPI (4′,6-diamidino-2-phenylindole; Vector Labs Inc., CA) stained coronal sections (40 μm thickness), cut on a freezing microtome and mounted on microscope slides were digitally scanned at 40× magnification (Nanozoomer, Hammatsu Photonics, Japan). The images were transferred to Image J software (NIH, USA) and the lesion volumes were quantified. Volumes were measured by tracing lesion borders then multiplying the lesion area by section thickness and number of sections in the series. Lesion widths and depths were determined by superimposing a straight line connecting lesion boundaries and measuring in horizontal and vertical directions respectively.

Transcranial Direct Current Stimulation.

The tDCS stimulation protocol, consisted of 10 minutes of an anodal 65 μA direct current with an additional 30 ms of 65 μA pulses applied every 5 seconds. The pulses were included in the protocol in order to increase coherence between hemispheres. The stimulation was applied only during first week of rehabilitation concurrently with skilled reaching task. Rats in all groups were connected to the stimulator during all reaching trials, but the stimulator was switched on only for the Stim+stroke group.

Acute Surgery and Recordings.

Detailed descriptions of the surgery and recording procedures have been published. Briefly, after the last session of re-testing the animals were anaesthetized with urethane (1.5 g/kg), injected i.p. Rats were placed in a stereotaxic frame, and two 3 mm² craniotomies were prepared over the forelimb somatosensory cortex of each hemisphere (AP: +2.7 to −0.3; ML: ±2.5 to 5). One rat from the Stroke+Stim group died during the electrophysiological recordings and data from this experiment was excluded from analyses. Extracellular signals were recorded with silicon probes (NeuroNexus Technologies, Ann Arbor, Mich.) consisting of eight shanks (FIG. 9B), each shank separated by 200 μm. The location of the recording sites was determined to be layer V by histological reconstruction of the electrode tracks and electrode depth. Local field potentials were digitized and amplified using Digital Lynx system (Neuralynx, Bozeman, Mont.). Ten minutes of spontaneous activity was recorded followed by 20 min of electrical stimulation with the same parameters as in training (FIG. 9). This was followed by 10 mins of recording of spontaneous activity. At the end of the acute electrophysiological recordings animals were deeply anaesthetized with pentobarbital and perfused in order to perform histological analysis on the extracted brains using DAPI mounting medium.

Coherence Analysis.

Coherence was calculated as the magnitude squared coherence estimate using Welch's averaged, modified periodogram method implemented in Matlab (MathWorks, Inc.; mscohere function). The magnitude squared coherence estimate is a function of frequency with values between 0 and 1 that indicates how well input signals x and y correspond to each other at each frequency. The magnitude squared coherence C_{xy}(f) is a function of the power spectral densities (Pₓₓ(f) and P_{yy}(f)) of x and y and the cross power spectral density (P_{xy}(f)) of x and y: C_{xy}(f)=|P_{xy}(f)|/(Pₓₓ(f)·P_{yy}(f)).

### Results

Decrease in Local Field Potential (LFP) Coherence after Stroke.

To investigate how interactions between sensory areas change after stroke and how this is modified by the application of electrical stimulation three groups of rats were studied: Control (n=5), Stroke (n=5), and a group with stroke treated with direct current stimulation (Stroke+Stim., n=5, see Materials and Methods for details). All rats were implanted bilaterally with stimulating electrodes anterior to the cortical motor areas and one reference electrode over the cerebellum (FIG. 9A, B), but stimulation was applied only in the Stroke+Stim group (FIG. 9D, E; Materials and Methods). During the same surgery, focal cortical ischemia was induced by photothrombosis in the forelimb area of motor cortex in the hemisphere contralateral to the preferred paw in the Stroke+Stim and Stroke groups (Materials and Methods). Ischemia was largely constrained to cortical areas (FIG. 9B). Average infarct volumes per group (Vₛₜᵣₒₖₑ=5.42 mm³±2.01; V_{Stroke+Stim}=4.92 mm³±1.80) were not statistically different (p=0.9, t-test).

Persistent effects of stroke and electrical stimulation on cortical activity were measured after 8 weeks following infarct. Animals were anesthetized with urethane and LFPs were recorded in deep cortical layers. Two electrodes with 8 recording shanks each were inserted in sensory areas in both hemispheres (FIG. 9C, Materials and Methods). LFP power spectra in all experimental groups were highest around 0.5-2 Hz with a systematic decrease in higher frequencies. There were no significant differences between experimental groups (p_{contr-strok}>0.2; p_{contr-strok+stim}>0.2; p_{Stroke-Strok+Stim}>0.1; Kolmogorov-Smirnov test) or between lesion and intact hemispheres (p>0.2; Kolmogorov-Smirnov test) at any frequency band between 0.5-40 Hz.

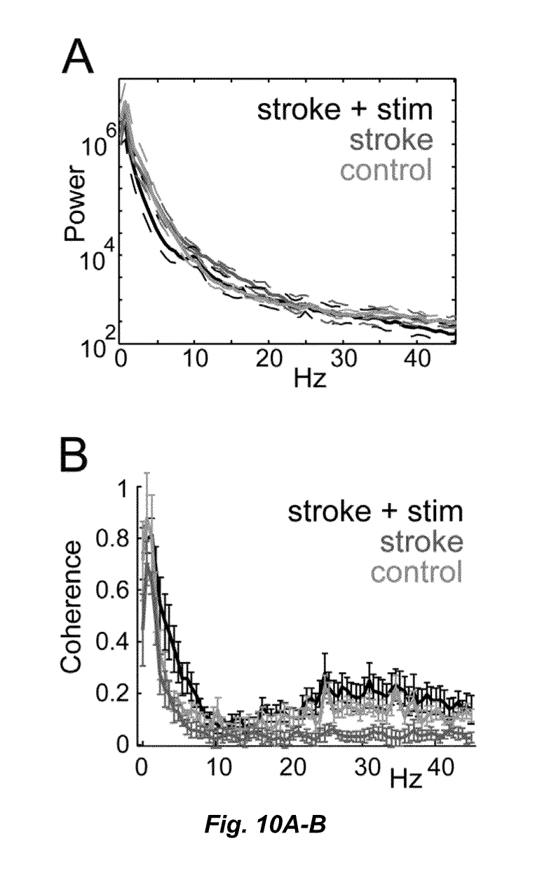

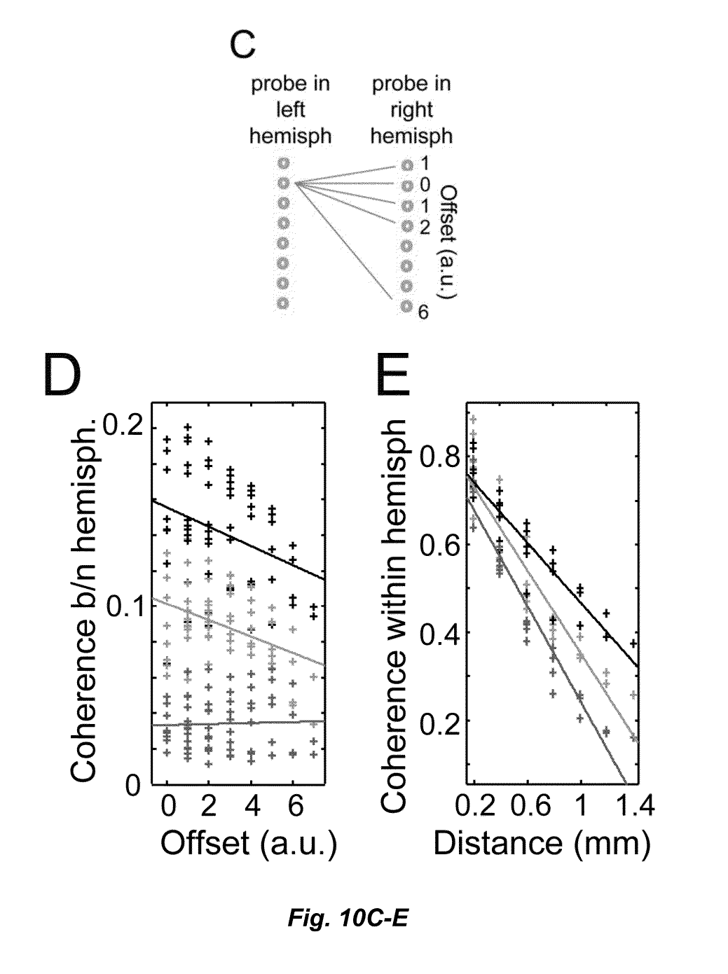

Despite the lack of differences in LFP power spectra, LFP coherence showed notable differences between groups (FIG. 10B) which were most pronounced in the low gamma frequency range (20-40 Hz). Inter-hemispheric coherence was significantly lower in the Stroke group as compared to the Control and Stroke+Stim groups (p_{Control-Strok}=0.044; p_{Stroke+Stim-Strok}=0.032; Kolmogorov-Smirnov test). This effect was consistent across different pairs of electrodes although values of coherence systematically changed with distance. For example, coherence between hemispheres was usually the highest between homologous areas and decreased with spatial offset between areas (FIG. 10C, D; R_{contr}=−0.41; p<0.001; R_{Stroke+Stim}=−0.31; p=0.012; R_{Stroke}=0.03; p=0.78).

In the Stroke group the correlation was not significant, most likely due to insufficient coherence to detect this effect. Similarly, within-hemisphere coherence decreased significantly as a function of distance between electrodes (FIG. 10E; R_{Contr}=−0.93; p<0.001; R_{Stroke+Stim}=−0.92; p<0.001; R_{Stroke}=−0.96; p<0.0001). There were no systematic differences between the intact and lesion hemispheres concerning relation between coherence and distance. Altogether, these results suggest that focal ischemia reduces the coherence between cortical areas, and electrical stimulation restores coherence in the gamma band.

Coherence in Gamma Band Correlates with Behavior.

A skilled forelimb reaching task was used to provide a behavioral measure of motor recovery after stroke (FIG. 11A, Materials and Methods). On the last day of testing (one day prior to electrophysiological recordings), reaching success (see Materials and Methods) was calculated for each animal (RS_{Control}=43.76±11.05 SEM, RS_{Stroke}=20.92±6.31 SEM, RS_{Stroke+Stim}=40.81±8.3 SEM). These results are consistent with previous studies showing decreased reaching success in rats after focal ischemia and improvements due to anodal electrical stimulation. Reaching success and coherence in gamma band were strongly correlated (FIG. 11B; R=0.78; p=0.001). Notably, even within individual experimental groups correlations had the tendency to be positive, suggesting a general positive relationship between coherence and skilled movements (R_{Contr}=0.83; p=0.08; R_{Stroke+Stim}=0.75; p=0.24; R_{Stroke}=0.18; p=0.77). These findings indicate that gamma band coherence may be important for competency in motor tasks.

Anodal Stimulation Increases LFP Coherence.

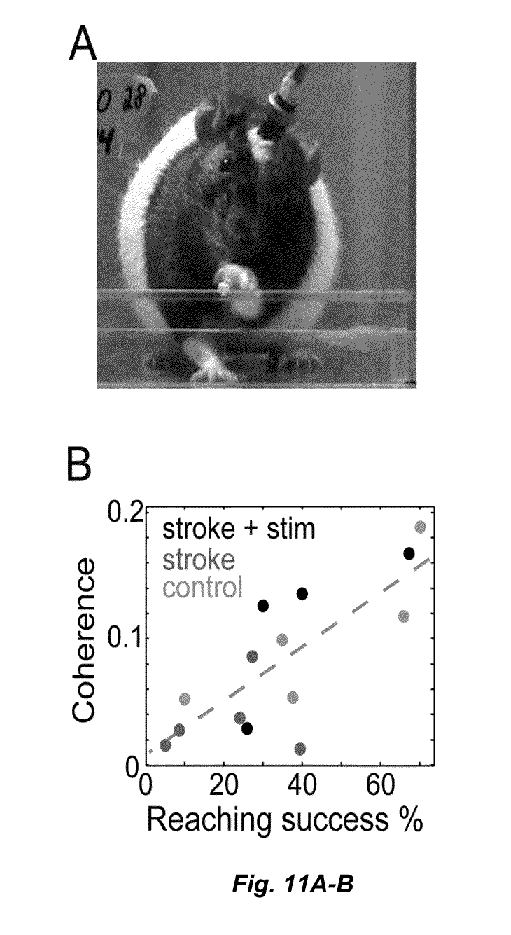

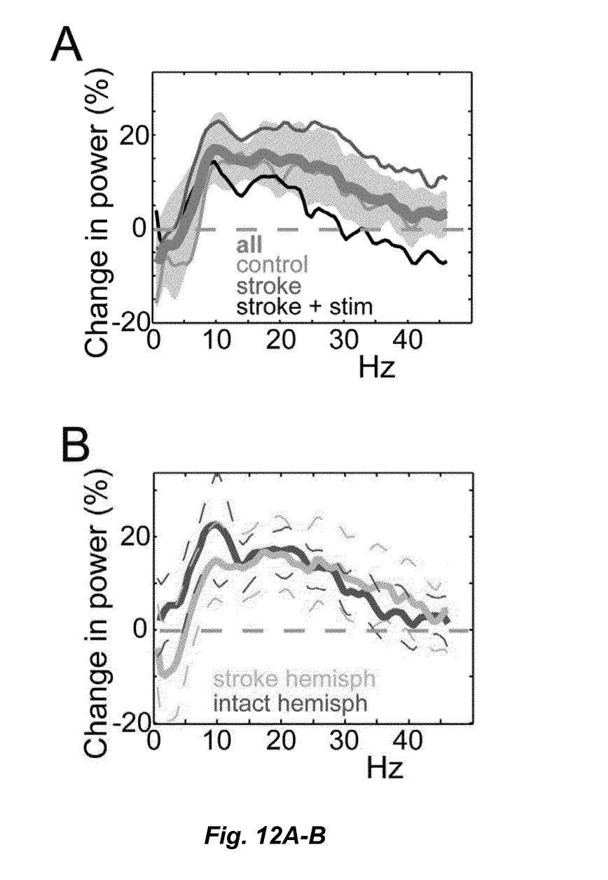

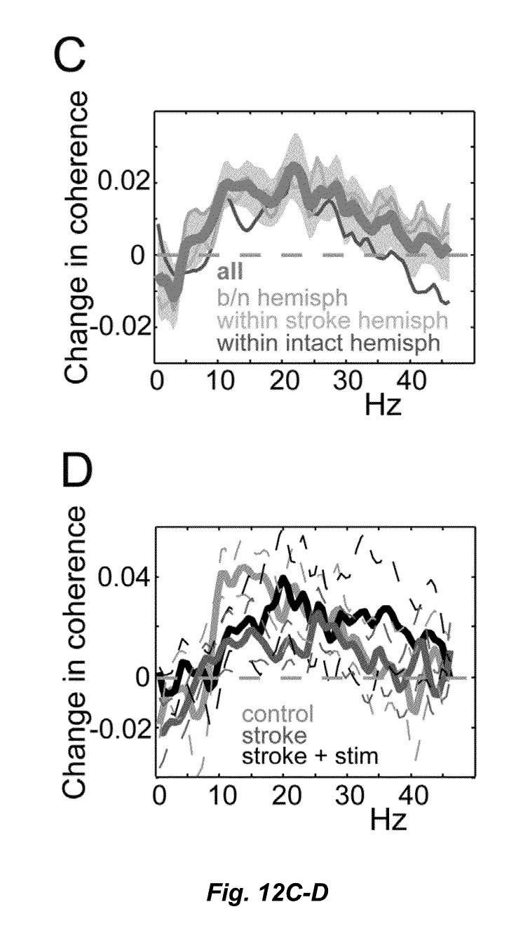

The data above shows that the Stroke+Stim group had higher gamma band coherence. To investigate if enhanced gamma band coherence is causally linked to electrical stimulation Applicant examined the effect of electrical stimulation on brain activity. During LFP recordings in anesthetized animals Applicant applied 20 min of anodal stimulation in order to compare brain activity directly before and after electrical stimulation (Materials and Methods). The stimulation significantly increased LFP power in the 10-40 Hz band (p=0.016; t-test) and this effect was consistent across all experimental groups (FIG. 12A). Moreover, LFP power increased similarly in the intact and lesion hemispheres (FIG. 12B; p=1; paired sign test between intact and stroke hemisphere in 10-40 Hz band).

Importantly, increased LFP power after electrical stimulation was associated with increased coherence in the corresponding frequency band (FIG. 4C; p=0.038; t-test; 10-40 Hz). Again, this increase was consistent within the intact and lesion hemispheres and between hemispheres (FIG. 12C). This effect was similar across experimental groups (FIG. 12D), indicating that anodal stimulation is directly causing a general increase in coherence among cortical areas.

Discussion.

In this study Applicant performed electrophysiological recordings to investigate interactions between cortical networks in rats treated with tDCS after focal ischemic motor cortex lesion. Applicant described a widespread decrease in gamma band coherence between cortical areas following ischemia. Applicant also showed that coherence in gamma band range was positively correlated with performance on a skilled motor task. Importantly, Applicant demonstrated that anodal stimulation enhanced gamma band coherence between cortical areas, which may explain the therapeutic effect of tDCS on stroke recovery. Our results indicate that designing electrical stimulation protocols that maximize synchronization among cortical areas can facilitate neuroplastic mechanisms and provide more effective stroke therapies.

Although very few studies investigated changes in gamma band coherence after stroke, its decrease relates to a general reduction in connectivity among brain areas following stroke. Other studies, using data from diffusion magnetic resonance imaging, compared connectivity between 56 brain areas in stroke patients and healthy controls, and showed decreased communication among a number of brain regions in stroke patients. Similar results were obtained using near-infrared spectroscopy showing that the interhemispheric correlation coefficient at frequency bands 0.15-3 Hz was smaller in stroke patients. Consistent with these results is the reduction in interhemispheric functional connectivity in a rat model of stroke using low-frequency BOLD fluctuations.

This reduction was also correlated with decreased sensorimotor function. Similarly, analysis of functional connectivity with EEG also revealed reduced interhemispheric coherence in stroke patients. Based on analysis of the topological configuration of the resting-state networks, brain networks shifted toward a more random and less optimized mode of function after stroke. Thus our finding of decreased coherence in gamma band are another manifestation of reduced efficiency of communication among brain networks after stroke.

Cortical gamma activity has been implicated in a variety of functions including attention, working memory, object recognition, feature binding, information processing and sensorimotor performance. In general, gamma activity is associated with top-down attentional processing by which the brain may be controlling the flow of sensorimotor information. Our results correspond well to this general framework. It should be noted that our electrophysiological data were recorded under anesthesia which means observed changes reflect long-lasting properties of cortical circuitry rather than task-related changes in neuronal synchronization. Thus, the higher gamma activity observed in rats with better skilled movements indicate that cortical networks in those animals can synchronize more readily in the gamma range, which contributes to better control over motor tasks. Such analysis is supported by experiments demonstrating that higher baseline levels of gamma activity correspond to shorter response times, and task performance efficiency.

In this study, Applicant show that anodal stimulation enhances gamma activity. This is of particular interest because the electrophysiological effect of direct current stimulation on neuronal activity in vivo is poorly understood. To our knowledge this study presents the first evidence of an effect of direct current stimulation on gamma oscillations. Interestingly, the increase in power above 10 Hz also resembles characteristics of an activated state. For example, during slow wave sleep or during restful wakefulness, cortical activity is dominated by slow rhythms <3 Hz characteristic for inactivated brain state. Conversely, during the attentive state, brain activity decreases in the low frequencies with a simultaneous increase in gamma range, which characterizes the activated state and is also linked to attention. Thus, increasing gamma activity with anodal stimulation can emulate the effect of attention.

This idea is consistent with other studies in which the application of anodal tDCS had a striking resemblance to the effects of increased attention. For example, anodal tDCS applied over the prefrontal cortex can improve working memory, tDCS applied over the temporal lobe facilitates problem solving and motor performance is improved when tDCS is applied over the motor cortex. Therefore, the effect of tDCS is linked enhanced attention, and the use of tDCS thus leads to a better conceptual understanding of tDCS mechanisms and to better treatment strategies. It is also worth to note that stroke patients show attention deficits. Thus, the therapeutic effect of tDCS when combined with physical rehabilitation can work by helping motor networks to “pay attention” to an exercise, thereby enhancing its efficacy.

From a translational research perspective, our data indicates that by identifying the parameters of electrical stimulation that maximize gamma coherence, the data can lead to more effective stroke therapies. In this study, Applicant used constant anodal stimulation at 65 μA with additional 30 ms long 130 μA pulses at 0.2 Hz applied over both hemispheres. This stimulus was chosen because anodal currents will excite motor cortices and additional short pulses can assist synchronization between hemispheres. However, other types of stimuli could also be designed to activate and synchronize cortical networks effectively, leading to better stroke therapy. To our knowledge, our study provides the first guidelines for improving tDCS effectiveness based on electrophysiological data.

In summary, our findings show that improved coherent activity between neuronal populations facilitates recovery after ischemic lesion. Importantly, our study indicate that tDCS mediates sensorimotor recovery by increasing the synchronization between cortical networks.

### Example 3

### Improving Stroke Recovery Therapy

Following a stroke, patients often must undergo physical and/or mental therapy to regain lost functional ability. Typically, rehabilitation is a slow process requiring daily training over a period of months or years. Embodiments of the present disclosure can significantly reduce the time and effort necessary to achieve similar or improved results as comparing to standard therapy (which is currently administer during times which may not be optimal for brain modification). For example, a patient can be configured to wear an EEG-based device to monitor brain state during rehabilitation. An EEG-based device can provide information regarding optimal times for training during which the brain is in the most desynchronized (plastic) state. This information can be displayed to a patient in, for example, a numerical score ranging from 1 to 10 (where 10 indicates most desynchronized brain state). Thus, a patient can chose to perform the most difficult exercises during times of highest brain desynchronization, while choosing to rest or to perform less demanding tasks when the brain is in a less desynchronized state, a situation where therapy would be less effective. This personalized therapy during periods of highest brain desynchronization can significantly improve rehabilitation recovery over current techniques.

### Example 4

### Optimizing tDCS Effect for Stroke Recovery Therapy

Transcranial direct current stimulation (tDCS) is a valuable technique to assist with treatment of a wide range of neurological conditions, including stroke. This technique relies on the application of weak electrical currents (in amounts from about 0.1 mA to about 2 mA as described previously) through the scalp, and can modulate neuronal activity in the brain (FIG. 9A). Experimentation shows that tDCS can increase the level of brain desynchronization (FIG. 12), which state can be used as an electrical biomarker for brain plasticity. Currently the optimal parameters for tDCS (e.g. amount of current, exact electrode placement) vary across patients and can be determined empirically or on a patient-to-patient basis. Embodiments of the current disclosure can optimize tDCS parameters for each patient by using an EEG-based device for measuring brain desynchronization. For example, patients with a stroke in the brain motor cortex may adjust placement of tDCS electrodes and the applied amount of current to maximize brain desynchronization over the motor cortex as measured by the EEG-based device. Thus, maximizing desynchronization over the affected area can result in increased local brain plasticity. This can result in faster and more effective stroke rehabilitation for each patient.

### Example 5

### Optimizing Timing for Learning Mental or Motor Skills

Related to choosing the most appropriate timing for rehabilitation therapy as described above, a similar method can be used in healthy subjects to determine an optimal time for learning. For example, students who need to learn new material (e.g., language or mathematics) may chose when to study based on measuring brain desynchronization. Accordingly, the student can focus on the most difficult material to learn when the brain is in the highest desynchronized brain state. An EEG-based measuring device can provide a clear indication of when the brain is in the highest desynchronized brain state. Conversely, students may choose to rest or switch topics when the brain is in a less desynchronized state. This method can significantly improve effectiveness of learning new material. Likewise, learning a new physical skill (e.g. throwing a baseball or other athletic tasks) can be optimized by choosing to train during periods of highest brain desynchronization.

### Example 6

### Pre-Screening Potential Compounds for Drugs Development

Testing potentially new neurological drugs is a very lengthy process, often requiring years of animal trials where the effectiveness of a drug must be estimated from behavioral improvements. Individual experiments in the process may take weeks to months to complete. Because most neurological improvements from disorders or injury require changes in neuronal connectivity, a more plastic brain state can facilitate such changes. Therefore, the process of drug development can be significantly improved using brain desynchronization as a measure of drug effectiveness. The effect of the drug in producing a higher desynchronized brain state can be initially tested, thereby determining the most prominent potential drug compounds for improving, for example, stroke recovery. Thus, for instance, rats can be injected with each compound and changes in brain activity can then be measured continuously over periods of time (up to few hours or days). Only compounds that induce the highest desynchronized (plastic) brain state would be recommended for future trials. This new method can reduce the time needed for pre-screening drugs from months to hours or days.

### Example 7

### Treating Neurological Disorders (e.g. Depression, Brain Stroke, Etc.)

Finding the right medication to treat a neurological disorder can be a complicated process because each patient can respond differently to a given treatment. The problem with current treatments (e.g., for depression or epilepsy) is that patients are administered a medication for multiple weeks before it can be determined if this particular medication, or combination of medications, shows positive results for that patient. Using an embodiment of the present disclosure, this process can be improved by measuring brain activity with an EEG-based device for relatively short period of time (hours to days). If a given medication does not induce a change in brain desynchronization after a single or multiple doses, then it will be unlikely to affect behavior (i.e., mood in the case of antidepressant, motor recovery in case of brain stroke). In such cases, the patient can be quickly and easily switched to a different medication. Moreover, drug treatment of many neurological disorders is often combined with psychotherapy (e.g., for depression or PTSD) or physiotherapy (e.g., for stroke) aimed at changing a patient's behavior or ability to control behavior. Thus, choosing a medication based on its increasing or decreasing effect on brain desynchronization (plasticity) can provide a faster and more effective means of finding the right treatment, or a combination of treatments, for a particular patient.

### Example 8

### Optimizing Brain State for Learning or Rehabilitation

As discussed above, brain desynchronization can be measured to determine when the brain is most plastic and thus most ‘receptive’ to learning or rehabilitation. In some embodiments of the current disclosure, a person may also use an EEG-based device to measure instantaneous brain desynchronization and use it to learn to self-induce a more desynchronized state. This can be useful when a person may need to learn new material or skills. For example, a person having an instantaneous display of their own brain desynchronization state (e.g. on scale 1-10, with 10 indicating most desynchronized/plastic brain state) may learn within minutes or hours how to induce a mental state with the highest levels of brain desynchronization. This ability to learn to control some aspects of brain signals is known as biofeedback. Therefore, by measuring when the brain is most desynchronized, a person can self-train to induce a higher level of brain desynchronization by altering activity levels, providing a stimulus, or changing thought patters, and thereby improve learning of mental or motor skills instead of waiting for brain desynchronization to randomly occur.

### Example 9

### Optimizing Brain State for Sleep, Relaxation, and Anxiety Mitigation

In some instances, reduced brain activation is desirable, such as when relaxing, using ‘mindfullness’ techniques for stress/anxiety reduction, or for inducing sleep. These states (stress, anxiety, wakefulness, etc.), as well as anesthesia, are associated with an increase in brain synchronization (FIG. 1a,c). Thus, using an EEG-based device and an instantaneous display of brain desynchronization, a person can learn to increase levels of brain desynchronization by altering activity levels, providing a stimulus, or changing thought patters when the brain is experiencing an increase in synchronization. This can result in ‘quieting’ of the brain and can facilitate relaxation, sleep, and reduced stress/anxiety.

### Example 10

### Brain State Dependent Therapy for Improved Neural Training and Rehabilitation

Described herein is a novel process for improved efficacy of neural training in healthy subjects and rehabilitation in subjects with neurological disorders. This disclosure was based in part on results from animal studies from our lab (Bermudez et al., Neuron, 79, 555-566, 2013). This example provides results from human subjects. Using memory task and EEG recordings to simultaneously monitor brain activity, Applicant showed that when the brain is in more desynchronized state, subjects were able to memorize (encode) more information. Thus, consistent with the animal studies, brain plasticity in humans is correlated with brain desynchronization. This provides additional strong evidences for validity of improved neural training and rehabilitation as herein.

For the experiment, Applicant recruited undergraduate students from University of Lethbridge who signed an informed consent form. The participants performed a word-pair matching task (FIG. 13). The task was modified from Mander et al. (Nature Neurosci., Advance Online Publication, 27 Jan. 2013) and uses word-nonsense word pairs to maximize demands on novel episodic memory. The words and non-words were based on Buchanan et al. (Behay. Res., 2013, 45:746-757) and Rastle et al. (Quar. J. Exp. Psy. 2002, 55A, 1-24), respectively. The experiment was comprised of two phases. The learning phase consisted of four blocks of 20 word pairs each which was displayed on the monitor for five seconds (FIG. 13), and between each word pair there was a one second gap. In the testing phase, which was self-paced, the participants were asked to match each word with its non-word (FIG. 13). EEG was recorded with 128 Ag/Ag—Cl electrodes in an elastic net (Electrical Geodesics Inc., Eugene, Oreg., USA) as described in Ponjavic-Conte et al. (NeuroReport, 2012, 23(4), 240-245) (FIG. 14A), and the Desynchronization Index was calculated for each presentation of words. Behavioral data was also collected in order to measure the performance of the participants. The time between the learning phase and the testing phase was at least 30 seconds. The primary finding was that the performance and accuracy (e.g., lack of errors) of the participants in word-matching task increases if the word-pairs were presented during more desynchronized (plastic) state (p=0.0313; Wilcoxon signed-rank test) (FIG. 14B).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for assessing brain plasticity by measuring electrical brain biomarkers, the method comprising:
applying a biosensor to a patient, the biosensor measuring an electrical brain biomarker; and
receiving the measurement of the electrical brain biomarker by a measuring device, the measuring device recording the measurement of the electrical brain biomarker; and
sending the recording of the measurement of the electrical brain biomarker to a processor, the processor giving a near real-time analysis of the electrical brain biomarker, the near real-time analysis being an increase or decrease in the electrical brain biomarker, where the increase or decrease of the electrical brain biomarker is indicative of a state of brain plasticity in the patient in response to a stimulus, thereby assessing brain plasticity.

2. The method of claim 1 wherein the electrical brain biomarker is indicative of a desynchronized brain state characterized by an increase in power spectra output of brain electrical activity in gamma-band and a decrease in power spectra output in low frequency brain waves.

3. The method of claim 1, wherein the state of brain plasticity is determined by the ratio of power spectra output in the range of about 40 Hz to about 60 Hz to the power spectra output in the range of about 1.5 Hz to about 3.5 Hz, thereby providing a quantitative value for the state of brain plasticity.

4. The method of claim 1 wherein the stimulus is a drug administered to the patient.

5. The method of claim 1 wherein the stimulus is an electrical current.

6. The method of claim 1 wherein the patient is a human.

7. The method of claim 1 wherein the stimulus is continued if there is an increase in the patient's desynchronized brain state and where the stimulus is discontinued if there is a decrease in the patient's desynchronized brain state.

8. The method of claim 1 wherein the stimulus is selected from the group consisting of a visual, auditory, touch, taste or smell stimulus where the stimulus is continued if there is an increase in the patient's desynchronized brain state and discontinued if there is a decrease in the patient's desynchronized brain state.

9. A method for treating a neurological disease or trauma comprising:
applying a biosensor to a patient, the biosensor measuring an electrical brain biomarker;
receiving the measurement of the electrical brain biomarker by a measuring device, the measuring device recording the measurement of the electrical brain biomarker; and
sending the recording of the measurement of the electrical brain biomarker to a processor, the processor giving a near real-time analysis of the electrical brain biomarker, the near real-time analysis being an increase or decrease in the electrical brain biomarker, where a course of treatment is based on the increase or decrease of the electrical brain biomarker.

10. The method of claim 9 wherein the electrical brain biomarker is indicative of a desynchronized brain state characterized by an increase in power spectra output of brain electrical activity in gamma-band and a decrease in power spectra output in low frequency brain waves.

11. The method of claim 9, wherein the electrical brain biomarker is determined by a ratio of power spectra output in the range of about 40 Hz to about 60 Hz to the power spectra output in the range of about 1.5 Hz to about 3.5 Hz, wherein a larger ratio is indicative of a desynchronized brain state.

12. The method of claim 9 wherein the course of treatment is an administered drug regimen.

13. The method of claim 9 wherein the course of treatment is an electrical stimulus.

14. The method of claim 13 wherein the course of treatment is continued if there is an increase in a patient's desynchronized brain state and where the course of treatment is discontinued or reduced if there is a decrease in the patient's desynchronized brain state.

15. The method of claim 12 wherein the type of drug administered to the patient is changed based on the increase or decrease in desynchronized brain state.

16. The method of claim 12 wherein the drug dosage administered to the patient is changed based on the increase or decrease in desynchronized brain state.

17. The method of claim 12 wherein the duration of drug administered to the patient is changed bases on the increase or decrease in desynchronized brain state.

18. The method of claim 9 wherein the neurological disease or trauma is a neurodegenerative disease or a traumatic brain injury.

19. The method of claim 18 wherein the neurodegenerative disease is selected from the group consisting of Alzheimer's disease, Pick's disease, corticobasal degeneration, progressive supranuclear palsy, frontotemporal dementia, parkinsonism (linked to chromosome 17, FTDP-17), Parkinson's disease, diffuse Lewy body disease, brain stroke, amyotrophic lateral sclerosis, Niemann-Pick disease, Hallervorden-Spatz syndrome, Down syndrome, neuroaxonal dystrophy, and multiple system atrophy.

20. The method of claim 9 wherein the patient is a human.
